(19) United States
(12) Patent Application Publication
(10) Pub. No.: US 2024/0240201 A1
(43) Pub. Date: Jul. 18, 2024

(54) CELL CONVERSION

(71) Applicant: MOGRIFY LIMITED, Cambridge Cambridgeshire (GB)

(72) Inventors: David Anderson, Cambridge Cambridgeshire (GB); Rodrigo Luiz Dos Santos, Cambridge Cambridgeshire (GB)

(21) Appl. No.: 18/289,516

(22) PCT Filed: May 4, 2022
(86) PCT No.: PCT/GB2022/051129

§ 371 Date: Nov. 3, 2023

(30) Foreign Application Priority Data
May 4, 2021 (GB) 2106360.7
Aug. 4, 2021 (GB) 2111278.4

(51) Int. Cl. C12N 15/86; C12N 5/071; C12N 5/077; C12N 5/0786; C12N 5/0793

(52) U.S. Cl. CPC C12N 15/86; C12N 5/0619; C12N 5/0645; C12N 5/0658; C12N 5/067; C12N 2506/1307; C12N 2506/45; C12N 2770/36143; C12N 2840/203

(57) ABSTRACT

Described herein are methods and compositions containing self-replicating RNA encoding at least one transcription factor and their uses in cell conversion of a pluripotent stem cell. a multipotent stem cell. or a differentiated source cell to a desired target cell. The methods and compositions can be used to transfect human pluripotent or multipotent stem cells and various human somatic cell types with self- replicating RNA to convert cells in vitro, in vivo or ex vivo.

### FIELD OF THE INVENTION

The present invention relates to methods and compositions containing self-replicating RNA encoding a transcription factor and their uses in cell conversion of pluripotent stem cells, multipotent stem cells or differentiated source cells to a desired target cell.

### BACKGROUND TO THE INVENTION

There is a need for alternative means of replacing cells without having to transplant an organ. Cell-based regenerative therapy requires the generation of specific cell types for replacing tissues damaged by injury, disease or age. Human embryonic stem cells (hESCs) have the potential to differentiate into every cell type in the human body and have therefore been extensively studied as a source for replacement therapy. However, hESCs cannot be derived in a patient-specific fashion since they are established from cultured blastocysts. Therefore, immune rejection and ethical concerns are the main barriers that prevent the transfer of hESC technology to clinical applications. In 2007, it was reported that four transcription factors could be used to convert differentiated cells back to a pluripotent stem cell-state (Takahashi et al.,2007 Cell, 131(5), 861-872; doi.org/10.1016/j.cell.2007.11.019). These transcription factors were Klf4, Sox2, Oct4 and cMyc, and due to the induced nature of the pluripotent state, these cells have been called induced pluripotent stem cells (iPSCs). Transcription factors have also been shown to enable the conversion of one differentiated cell type to another without going through a pluripotent state, a process known as cell conversion, transdifferentiation or forward programming. It is therefore possible to switch the phenotype of one differentiated cell type to another, both in vitro and directly in vivo, however the elements required for cell conversion are difficult to identify, and, in most instances, are unknown.

The present inventors are able to efficiently identify transcription factors for converting one cell type to another using their proprietary technology (WO2017/106932). An example of a cell conversion in relation to this invention is the conversion of fibroblasts to myoblasts, achieved by exogenously expressing MYOD in fibroblasts.

Typically, source cells are converted into target cells by the use of DNA, Adeno associated virus (AAV) or lentivirus engineered to encode the identified transcription factors. Although success has been achieved with these approaches, a major disadvantage of these methods is the potential for DNA integration into the host cell genome, which can lead to malignancy and other unwanted side-effects.

In vitro transcribed messenger RNA (mRNA) has also been successfully employed in cell conversion studies, but it is rapidly degraded in recipient cells and multiple transfections of source cells are often needed for successful conversion (Akiyama et al., 2020 Stem Cells Translational Medicine; doi.org/10.1002/sctm.20-0302). This limits its utility for cell conversion.

A self-replicating RNA is an RNA element that encodes sequence and proteins that allow the RNA to transcribe and replicate itself when introduced into a host cell. The indicated sequences and proteins are typically of viral origin, derived from single strand RNA viruses, including but not limited to alphaviruses, picornaviruses, flaviviruses, rubiviruses, pestiviruses, hepaciviruses, filoviruses, caliciviruses and coronaviruses. Specifically, elements from the alphaviruses Semliki Forest virus (SFV), Sindbis virus (SINV) and Venezuelan Equine Encephalitis virus (VEEV) are routinely incorporated into self-replicating RNA. The alphavirus elements required to allow an RNA element to self-replicate include the non-structural proteins (nsP1-4), 5′ genomic UTR element, subgenomic promoter, and 3′ genomic UTR element of the virus. To be of functional use, the elements allowing replication of the RNA must also permit expression of a target protein.

It has been demonstrated that self-replicating RNA encoding known combinations of reprogramming factors can be employed to convert terminally differentiated cells back to a pluripotent state (Yoshioka et al., 2013 Cell Stem Cell, 13(2), 246-254; doi.org/10.1016/j.stem.2013.06.001; Yoshioka & Dowdy, 2017 PLOS ONE, 12(7), 1-17; doi.org/10.1371/journal.pone.0182018). Adult fibroblasts were converted to iPSCs using self-replicating RNA encoding a combination of reprogramming factors comprising at least four factors: POU5F1, SOX2, KLF4 and at least one from c-MYC, GLIS1 and LIN28 (Yoshioka et al., 2013; Yoshioka & Dowdy 2017). Once reprogrammed back to a pluripotent state with the reprogramming factor self-replicating RNA, the newly derived pluripotent stem cells can be subsequently differentiated to a target desirable cell type. Further, it has been demonstrated that transdifferentiation of differentiated cells can be achieved by transiently expressing compositions of the above detailed reprogramming factors in combination with specific growth media or transcription factors required for specific target cell conversions (Efe et al., 2011 Nature Cell Biology, 13(3), 215-222; doi.org/10.1038/ncb2164). This method of reprogramming factor-mediated transdifferentiation is thought to induce the early stages of the reprogramming process and erase cell identity by epigenetic mechanisms, making cells more permissive to differentiating to target cell lineages.

There remains a need for efficient delivery of genes that can be expressed in a cell to permit the stable transformation or conversion of a cell from one type to another desired target cell.

### SUMMARY OF THE INVENTION

The invention relates to the use of self-replicating RNA (repRNA) for the expression of factors required for cell conversion. In a first embodiment the present invention provides a self-replicating RNA composition comprising a sequence encoding a transcription factor or variants thereof, that converts a pluripotent stem cell (for example an induced pluripotent stem cell), a multipotent stem cell, or a differentiated source cell (for example a fibroblast), to a cell exhibiting at least one phenotypic characteristic of a differentiated target cell (i.e. a different differentiated target cell when the source cell is a differentiated cell), without the use of transcription factors known to promote the reprogramming of differentiated cells to a pluripotent state.

In a second embodiment there is provided a method for converting a pluripotent stem cell, a multipotent stem cell, or a differentiated source cell to a cell exhibiting at least one phenotypic characteristic of a differentiated target cell, wherein the source cell does not exhibit the phenotypic characteristic of the differentiated target cell, the method comprising expressing at least one transcription factor by introducing a self-replicating RNA encoding a transcription factor or transcription factors into the pluripotent stem cell, multipotent stem cell or differentiated source cell, wherein the transcription factor or transcription factors are not a transcription factor known to promote the reprogramming of differentiated cells to a pluripotent state. The pluripotent stem cells are induced pluripotent stem cells or embryonic stem cells. The multipotent stem cells are neural stem cells (NSCs), mesenchymal stem cells (MSCs) or hematopoietic stem cells (HSCs). The differentiated source cells can be any differentiated cells, typically a primary cell such as myocytes, adipocytes, melanocytes, osteoblasts, oligodendrocytes, neurons, endothelial cells, pancreatic beta cells, hepatocytes, cardiomyocytes, fibroblasts, T cells, natural killer (NK) cells, macrophages, dendritic cells, epithelial cells, retinal pigment epithelium cells, Müller glia cells, or myoblast cells. T cells, NK cells, macrophages and other lymphocytes can be isolated cells from peripheral blood mononuclear cell (PBMC) fractions such as CD4+lymphocyte; CD8+lymphocyte; CD56+NK cell or a CD19+B lymphocyte. The differentiated target cells can be any desirable differentiated target cell, typically a therapeutic cell such as myocytes, adipocytes, melanocytes, osteoblasts, oligodendrocytes, neurons, endothelial cells, pancreatic beta cells, hepatocytes, cardiomyocytes, T cells, NK cells, macrophages, dendritic cells, epithelial cells, retinal pigment epithelium cells, photoreceptors, myoblast cells, or progenitor cells amongst others.

In particular embodiments the differentiated target cell is a neuronal cell, a myocyte, a macrophage or a hepatic progenitor cell.

In one aspect of the invention the self-replicating RNA is derived from a single stranded RNA virus devoid of the RNA virus structural genes and encodes a transcription factor, such that it can be expressed.

In an aspect of the invention, the self-replicating RNA contains one or more sequences (in addition to the transcription factor or transcription factors) encoding for two, three, four, or more open reading frames, such as, transcriptional modulators, fluorescent reporters (e.g. GFP, mCherry, BFP, mKate, etc), antibiotic resistance genes (e.g. puromycin, blasticidin, hygromycin, neomycin, zeomycin), or inhibitors of type I and/or type III interferon immune responses (such as the vaccinia virus proteins E3, K3, and B18R and the influenza virus protein NS1) (Beissert et al., 2017 Human Gene Therapy, 28(12), 1138-1146; doi.org/10.1089/hum.2017.121). In an embodiment, and advantageously for those source cells that require more than one transcription factor to ensure cell conversion into a differentiated target cell, the self-replicating RNA encodes more than one transcription factor. This ensures that the two or more transcription factors are delivered to the same cell. The self-replicating RNA encodes at least one, at least two, at least three, at least four, at least five or at least six transcription factors.

In a further aspect of the invention, one or more self-replicating RNAs encoding two or more transcription factors are delivered in a single step, in order to achieve cell conversion. Multiple transcription factors introduced in a single step transfection of source cells provides more efficient delivery of the transcription factors. A single step transfection also removes the need for any enrichment process for successfully transfected cells in a sequential transcription factor introduction method.

Advantageously, the present invention provides a method of transfecting human pluripotent stem cells and various human somatic cell types with the self-replicating RNA. Advantageously, in a population of cells the inventors demonstrate very high efficiency of expression of an open reading frame encoded by a self-replicating RNA. More than 70% of the cells as measured by expression of the fluorescent reporter protein GFP encoded in the self-replicating RNA were shown to express the protein. As shown in the examples, expression of a GFP reporter was detected for up to 2 weeks or more, which was substantially longer than observed with plasmid or mRNA-based expression of GFP.

Advantageously, this extended duration of expression is an important factor to ensure efficient cell conversion can be achieved, as cell conversion experiments typically require expression of transcription factors for greater than 7 days (Yoshioka et al., 2013). Advantageously, this extended expression duration observed with a single transfection of self-replicating RNA allows the avoidance of repeated transfection to achieve prolonged gene expression, as required for cell conversion with mRNA, and the avoidance of the use of lentivirus which integrates into host cell DNA, resulting in their genetic modification. Repeated transfections are technically challenging and can be detrimental to cell viability, and genetic modification is undesirable in therapeutic cell types. Advantageously, the present inventors have also demonstrated a higher efficiency of neuronal conversion of iPSC when cells were transfected with a self-replicating RNA compared to lentivirus or mRNA. The methods and compositions of the present invention can be used to convert cells in vitro, in vivo or ex vivo.

Examples of human pluripotent stem cells are induced pluripotent stem cells (iPSCs) and embryonic stem cells (hESCs).

Advantageously, the present invention provides a method of transfecting human differentiated cells with the self-replicating RNA at high efficiency. For example, when the source cells are human dermal fibroblasts or iPSCs, more than 50%, 60% or 70% of the population express an open reading frame encoded by the self-replicating RNA. This has been demonstrated by expression of the fluorescent reporter protein GFP encoded in the self-replicating RNA and as compared to traditional mRNA approaches. Examples of human primary cells are dermal fibroblasts, human chondrocytes, T cells, NK cells, macrophages, dendritic cells, etc. Additionally, duration of expression was shown for up to 2 weeks or more. Such an increase in open reading frame expression efficiency demonstrates that the expression of a transcription factor will be sufficiently robust to allow for efficient transformation of a source cell into a cell exhibiting at least one phenotypic characteristic of a differentiated target cell.

It is possible to drive or initiate the process of transdifferentiation using pluripotency inducing reprogramming factors (Efe et al., 2011). Although the mechanism of reprogramming factor-mediated transdifferentiation is not fully understood, the use of pluripotency inducing factor combinations to induce the early stages of the reprogramming process in the differentiated source cell, either shortly before or during a transdifferentiation process, leads to the unfavourable possibility of generating oncogenic cells. Advantageously, the present invention provides a method of using self-replicating RNA to express transcription factors to directly transdifferentiate differentiated source cells to a different differentiated target cell, without the use of pluripotency inducing transcription factor combinations. This is demonstrated by the use of self-replicating RNA that encodes transcription factors outside of the pluripotency inducing transcription factor combinations (e.g. POU5F1+SOX2+KLF4+MYC; POU5F1+SOX2+NANOG+LIN28A; POU5F1+SOX2+KLF4+MYCL+LIN28A+GLIS1; POU5F1+SOX2+KLF4+MYC/GLIS1), for the cell conversion of differentiated source cells to different differentiated target cells. Preferably, the transcription factor(s) do not include more than one transcription factor from the list of known pluripotency inducing transcription factors consisting of POU5F1, SOX2, KLF4, MYC, MYCL, LIN28, NANOG and GLIS1. More preferably, the transcription factor(s) do not include any transcription factor from the list of known pluripotency inducing transcription factors consisting of POU5F1, SOX2, KLF4, MYC, MYCL, LIN28, NANOG and GLIS1. Still more preferably, the transcription factor is not GLIS1, or is not a combination of OCT4, SOX2, KLF4, and c-MYC or GLIS1.

In some embodiments, the efficiency of transfection of self-replicating RNA into pluripotent stem cells, and differentiated cells, and the durability of self-replicating RNA expression over time may be enhanced by the inhibition of cellular antiviral responses. These cellular antiviral responses include type I and/or type Ill interferon responses, which can be dampened by the use of small molecule inhibitors of the interferon response pathway, such as the small molecule JAK1/2 inhibitor Ruxolitinib, the use of recombinant viral proteins, such as vaccinia virus-derived B18R, or by expression of virus-derived proteins genetically encoded within the self-replicating RNA itself, such as the sequence of vaccinia virus protein B18R (Blakney et al., 2021 Molecular Therapy, 29(3), 1174-1185; doi.org/10.1016/j.ymthe.2020.11.011). Small molecule or protein inhibitors of the interferon response pathway include, but are not limited to, fedratinib, upadacitinib, filgotinib, baricitinib, deucravacitinib, ritlecitinib, decernotinib, RIG011, BX795, MRT67307 and Y136R. Cellular antiviral responses that can be manipulated, either by small molecules, gene knockdown or gene overexpression, to increase self-replicating RNA expression in pluripotent stem cells may include the RNAi pathway, endogenously expressed PKR, OAS/RNaseL, a stem cell-specific DICER isoform known as antiviral dicer, and a mechanism utilising endogenous retrovirus encoded reverse transcriptase. In some embodiments, the molecule utilised for modulating the cellular antiviral response is encoded within the self-replicating RNA, and includes, but is not limited to viral proteins (e.g. vaccine virus B18R and yaba virus-like Y136R), short hairpin RNA (shRNA) or short interfering RNA (siRNA) that target and result in reduced expression of immune response pathway proteins, antibodies engineered to block or degrade receptors (e.g. PROTACs) or signal transduction proteins in the immune response pathway, and natural or dominant negative versions of proteins as means of disturbing the interferon response pathway.

In a further embodiment, the self-replicating RNA composition comprises a 26S promoter.

In another aspect the self-replicating RNA is split into two molecules for example with the 5′ genomic

UTR element, the non-structural proteins (nsP1-4), and the 3′ genomic UTR encoded on one RNA molecule, and the 5′ genomic UTR element, the subgenomic promoter, the target ORF, such as one or more transcription factors, and the 3′ genomic UTR element encoded on a second RNA molecule. The function of the self-replicating RNA is split across the two RNA molecules: a first molecule comprising sequences encoding non-structural proteins, required for replication of self-replicating RNA, and a second molecule comprising a sequence encoding at least one transcription factor and structural elements within the RNA to allow replication, including the 5′ genomic UTR element, the subgenomic promoter and the 3′ genomic UTR element. The non-structural proteins of alphavirus-derived self-replicating RNAs assemble to generate a multi-enzyme replicas complex, which is capable of recognizing sequence elements within the self-replicating RNA replicon and replicating the entire RNA template. The replicase complex is also capable of replicating full-length and truncated RNAs in trans, and minimally requires a series of conserved sequence elements in these “trans-replicons” for their replication (Beissert et al., 2019 Molecular Therapy, 28(1), 119-128; doi.org/10.1016/j.ymthe.2019.09.009). The replicase activity can be supplied in a number of ways, including by delivery of a plasmid, an mRNA or a separate self-replicating RNA encoding the required non-structural proteins. Advantageously, this trans-replicating RNA system confers the separation of the viral-derived replicative proteins from the transcription factors that are to be expressed, which allows for increased therapeutic safety and increased control over transcription factor expression dynamics.

In some embodiments of the invention there is provided a method for converting a source cell selected from a pluripotent stem cell, a multipotent stem cell, or a differentiated cell to a differentiated cell exhibiting at least one phenotypic characteristic of a target cell, the method comprising transforming or transfecting the source cell with a self-replicating RNA encoding a transcription factor. In one embodiment, the method comprises transforming or transfecting the source cell with a self-replicating RNA encoding at least one, at least two, at least 3, at least 4, at least 5 or at least 6 transcription factors. The source cell may be a pluripotent stem cell, for example an induced pluripotent stem cell, multipotent stem cell, or a differentiated cell. For example a differentiated cell source cell can be any differentiated cell, but is typically a primary cell such as a fibroblast, e.g., dermal fibroblasts, chondrocytes, T cells, NK cells, macrophages, dendritic cells, endothelial cell, e.g., human umbilical vein endothelial cells, epithelial cell, or a myoblast cell T cells, NK cells, macrophages and other lymphocytes maybe an isolated cell from peripheral blood mononuclear cell (PBMC) such as CD4+lymphocyte; CD8+lymphocyte; CD56+NK cell or a CD19+B lymphocyte. Preferably, the source cell is a fibroblast or endothelial cell. More preferably, the source cell is a dermal fibroblast.

In some embodiments, the present invention provides a method of generating a population of target cells from a population of source cells transfected with a self-replicating RNA encoding at least one transcription factor.

Advantageously, a self-replicating RNA formulation is provided comprising a self-replicating RNA composition or a self-replicating RNA according to the present invention, and a lipid or a lipid nanoparticle. Preferably, the RNA is formulated in a lipid nanoparticle. The lipid may be a cationic lipid, or a liposome formulation of the cationic lipid N-[1-(2,3-dioleyloxy)propyl]-n,n,n-trimethylammonium chloride (DOTMA) and dioleoyl phophotidylethanolamine (DOPE). Preferably, the self-replicating RNA formulation is a cationic nano-emulsion.

In some embodiments, the present invention provides a method of generating a population of neuronal cells from a population of pluripotent stem cells, by introducing a self-replicating RNA encoding the transcription factor Neurogenin-2 (Aliases: NEUROG2, Atoh4, Math4A, NGN2, bHLHa8, ngn-2) into the pluripotent stem cells, thereby increasing the amount of Neurogenin-2 in the pluripotent stem cell population and culturing the pluripotent stem cells under conditions to allow at least 0.1% of the pluripotent stem cell population to be converted into neurons exhibiting at least one phenotypic characteristic of the differentiated target cell, such as NCAM expression. Preferably, at least 1% of the pluripotent stem cell population is converted into neurons exhibiting at least one phenotypic characteristic of the differentiated target cell, or more preferably at least 10%.

In another embodiment, the present invention provides a method of generating a population of neuronal cells from a population of fibroblasts, by introducing a self-replicating RNA or self-replicating RNAs encoding the transcription factors ASCL1, POU3F2, NEUROD1 and MYT1L into the fibroblasts, thereby increasing the amount of each transcription factor in the fibroblast population and culturing the fibroblasts under conditions to allow at least 0.1% of the fibroblast population to be converted into neurons exhibiting at least one phenotypic characteristic of the differentiated target cell, such as NCAM expression. Preferably, at least 1% of the fibroblast population is converted into neurons exhibiting at least one phenotypic characteristic of the differentiated target cell, or more preferably at least 10%.

In another embodiment, the present invention provides a method of generating a population of myogenic cells from populations of pluripotent stem cells or fibroblasts, by introducing a self-replicating RNA encoding the transcription factor MYOD into the pluripotent stem cells or fibroblasts, thereby increasing the amount of MYOD in the pluripotent stem cell or fibroblast population and culturing the pluripotent stem cells or fibroblast cells under conditions to allow at least 0.1% of the source cell population to be converted into myogenic cells exhibiting at least one phenotypic characteristic of the differentiated target cell, such as myosin heavy chain expression. Preferably, at least 1% of the source cell population is converted into myogenic cells exhibiting at least one phenotypic characteristic of the differentiated target cell, or more preferably 10%.

In another embodiment, the present invention provides a method of generating a population of macrophage-like cells from a population of fibroblasts, by introducing a self-replicating RNA or self-replicating RNAs encoding the transcription factors SPI1 and CEBPA into the fibroblasts, thereby increasing the amount of each transcription factor in the fibroblast population and culturing the fibroblasts under conditions to allow at least 0.1% of the fibroblast population to be converted into macrophage-like cells exhibiting at least one phenotypic characteristic of the differentiated target cell, such as CD11b expression. Preferably, at least 1% of the fibroblast population is converted into macrophage-like cells exhibiting at least one phenotypic characteristic of the differentiated target cell, or more preferably 10%.

In another embodiment, the present invention provides a method of generating a population of hepatic progenitor cells from a population of endothelial cells, by introducing a self-replicating RNA or self-replicating RNAs encoding the transcription factors HNF1A, HNF4A, ONECUT1 and FOXA3 into the endothelial cells, thereby increasing the amount of each transcription factor in the endothelial cell population and culturing the endothelial cells under conditions to allow at least 0.1% of the endothelial cell population to be converted into hepatic progenitors exhibiting at least one phenotypic characteristic of the differentiated target cell, such as albumin expression. Preferably, at least 1% of the endothelial cell population is converted into hepatic progenitors exhibiting at least one phenotypic characteristic of the differentiated target cell, or more preferably 10%.

In some aspects, the present invention provides a method for converting a source cell to a cell exhibiting at least one phenotypic characteristic of a differentiated target cell, wherein the source cell is in a living animal, and the method comprises administering a self-replicating RNA composition encoding at least one transcription factor or a self-replicating formulation. In some embodiments, the living animal is a mammal, preferably a human.

### FIGURES

FIG. 1. A schematic diagram illustrating the features of a plasmid DNA template used for the generation of self-replicating RNA comprising VEEV non-structural proteins nsP1-4 flanked by VEEV 5′ and 3′ cis active non-coding regulatory regions, a T7 promoter and 26S subgenomic promoter positioned upstream of a multiple cloning site.

FIG. 2. Protocol for generation of self-replicating RNA from a plasmid DNA template by in vitro transcription. Plasmids encoding self-replicating RNA replicons are linearised by Mlul restriction endonuclease, and then used as a template for in vitro transcription using a T7 DNA dependent RNA polymerase. Following RNA precipitation and resuspension, self-replicating RNA can be visualized by gel electrophoresis.

FIG. 3. Expression of a self-replicating RNA encoding GFP in iPSC. iPSC were nucleofected with a self-replicating RNA encoding GFP-IRES-Puro^{R} and analyzed at 24 hour post-transfection. FIG. 3A shows a flow cytometry plot demonstrating the efficient expression of GFP in iPSCs transfected with GFP-IRES-Puro^{R} repRNA (right panel) with 96.4% of iPSCs being GFP positive compared to iPSCs mock transfected with no repRNA (control). FIG. 3B shows microscopy imaging of iPSCs mock transfected with no repRNA (control) and iPSCs transfected with GFP-IRES-Puro^{R} repRNA.

FIG. 4. Expression kinetics of GFP delivered as self-replicating RNA, conventional in vitro transcribed mRNA, plasmid or lentivirus. FIG. 4A shows the percentage of GFP positive cells and FIG. 4B shows the intensity of GFP expression of the GFP positive cells as determined by flow cytometry over a time course of 21 days (MFI=mean fluorescence intensity).

FIG. 5. Expression kinetics of self-replicating RNA under selective pressure. The data summarizes time course flow cytometry of repRNA mediated GFP expression in iPSCs following transfection with GFP-IRES-Puro^{R} repRNA and subsequent treatment of cells with puromycin and recombinant B18R versus control with no additional treatment. The time course of GFP expression is compared to that achieved with lentivirus.

FIG. 6. Validation of GFP self-replicating RNA function in multiple cell types including iPSC, HEK293T and primary human dermal fibroblasts. Self-replicating RNA encoding GFP-IRES-Puro^{R} was transfected into iPSC, HEK293T and HDFs and cells were analysed by flow cytometry 24 hours after transfection against control cells mock transfected without repRNA (FIG. 6A). Expression of GFP in HDF transfected with a self-replicating RNA encoding GFP-IRES-Puro^{R} and cultured in media supplemented with recombinant B18R or ruxolitinib was assessed by flow cytometry over a time course of 14 days (FIG. 6B).

FIG. 7. Experimental system for validating cell conversion with self-replicating RNA. FIG. 7A shows a schematic diagram of a plasmid allowing in vitro transcription of a repRNA encoding NEUROG2 transcription factor and Puro^{R}-T2A-GFP (NEUROG2-iPTG) separated by an IRES sequence. FIG. 7B is a diagram detailing experimental timelines for cell conversion of iPSC to neuronal cells.

FIG. 8. NEUROG2 encoded by a self-replicating RNA drives a neuronal morphology in iPSC at day 2 and day 7 post-transfection. Left panel demonstrated iPSCs transfected with GFP-IRES-Puro^{R} repRNA and the right panel demonstrated iPSCs transfected with NEUROG2-iPTG repRNA.

FIG. 9. NEUROG2 encoded by a self-replicating RNA efficiently converts iPSC to neurons. iPSC were transfected with either a control GFP-IRES-Puro^{R} repRNA or NEUROG2-iPTG repRNA and maintained in culture until 7 days post-transfection. FIG. 9A illustrates the qPCR expression data. FIG. 9B shows a flow cytometry analysis, and FIG. 9C is an immunocytochemistry indicating expression of neuronal markers and successful conversion of iPSC to neuronal cells using NEUROG2-iPTG repRNA.

FIG. 10. Efficiency of NEUROG2 driven conversion of iPSC to neurons is more efficient when delivered as self-replicating RNA. Lentivirus and self-replicating RNA are able to deliver NEUROG2 to iPSC and convert them to neurons as evidenced by TUBB3 immunocytochemistry (FIG. 10A) and NCAM flow cytometry (FIG. 10B) compared to control cells transfected with GFP-IRES-Puro^{R} repRNA. Self-replicating RNA delivery of NEUROG2 results in an approx. 3-fold higher efficiency of neuronal conversion of iPSC when compared to lentivirus.

FIG. 11. A combination of ASCL1+POU3F2 (BRN2)+MYT1L+NEUROD1 encoded by self-replicating RNA converts dermal fibroblasts to neurons. Schematic of polycistronic ASCL1-POU3F2-NEUROD1 repRNA is shown (FIG. 11A). HDF were transfected with individual repRNA encoding ASCL1, POU3F2, NEUROD1 and MYT1L or a polycistronic repRNA encoding ASCL1, POU3F2 and NEUROD1 with a separate repRNA encoding MYTIL, and maintained in culture for 14 days post-transfection. Conversion to neurons was assessed by immunocytochemistry to detect TUBB3 (FIG. 11B) and NCAM flow cytometry (FIG. 11C) compared to control cells mock transfected without repRNA.

FIG. 12. MYOD1 encoded by a self-replicating RNA efficiently converts iPSC and dermal fibroblasts to myocytes. iPSC (FIG. 12A) and HDF (FIG. 12B), were transfected with a repRNA encoding MYOD1, maintained in culture for 7-10 days post-transfection and conversion to myocytes was assessed by immunocytochemistry to detect myosin heavy chain, compared to control cells mock transfected without repRNA.

FIG. 13. A combination of SPI1+ CEBPA encoded by self-replicating RNA converts dermal fibroblasts to macrophage-like cells. HDF were transfected with repRNA encoding SPI1 and CEBPA, maintained in culture for 10 days post-transfection and conversion to macrophages was assessed by flow cytometry (FIGS. 13A and 13B) and qPCR (FIG. 13C) compared to mock (no repRNA) and control (GFP repRNA) transfected cells. Flow cytometry plots in FIG. 13B display events gated from CD45+ cells in the equivalent plots of FIG. 13A.

FIG. 14. A combination of HNF1A+HNF4A+ONECUT1+FOXA3 encoded by a self-replicating RNA converts endothelial cells to hepatic progenitor cells. Schematic of polycistronic HNF1A-HNF4A-ONECUT1 repRNA is shown (FIG. 14A). HUVECs were transfected with a polycistronic repRNA encoding HNF1A, HNF4A and ONECUT1 with a separate repRNA encoding FOXA3, and maintained in culture for 14 days post-transfection. Conversion to hepatic progenitor cells was assessed by immunocytochemistry to detect albumin, compared to control cells mock transfected without repRNA (FIG. 14B).

FIG. 15. Multiple small molecules and recombinant proteins can support high levels of self-replicating RNA expression in dermal fibroblasts. FIG. 15A shows detection of GFP expression (percent GFP positive cells) by flow cytometry in dermal fibroblasts at day 7 post-transfection with GFP repRNA and treatment with indicated small molecules or recombinant proteins, compared to control cells transfected with GFP repRNA that received either no treatment or DMSO treatment. FIG. 15B shows example immunocytochemistry detection of myosin heavy chain expression in dermal fibroblasts at day 10 post-transfection with MYOD1 repRNA followed by maintenance in myocyte conversion conditions detailed, and treatment with indicated small molecules or recombinant proteins. Control is transfected with MYOD1 repRNA and treated with DMSO.

FIG. 16. Validation of a trans-replicating RNA system for GFP expression in iPSC. FIG. 16A is a schematic detailing the molecular cloning steps to generate two truncated trans-replicating RNA vector templates. FIG. 16B shows detection of GFP expression by imaging at 48 hr and flow cytometry at 96 hr post-transfection with TR1-GFP repRNA, either alone (control), or in combination with a mCherry expressing repRNA or nsP1-4 expressing mRNA.

### DETAILED DESCRIPTION

As used herein, a “transcription factor” refers to a protein whose function is to regulate the expression of a particular gene or genes in a cell and controls the rate of transcription of said gene's DNA to messenger RNA. Specific transcription factors regulate the expression of gene(s) that provide the phenotypic characterisation of the cell. Examples of specific relevant transcription factors are listed in Table 1, Table 2 and Table 3. In some embodiments the transcription factors are any one or more of the transcription factors listed in Table 1, Table 2 or Table 3. In other embodiments, the transcription factors are any one or more of the transcription factors listed in a single row in Table 1, Table 2 or Table 3. In some embodiments, all of the transcription factors listed in a single row of Table 1, Table 2 or Table 3 may be used. The ‘+’ in Table 1, Table 2 or Table 3 denotes ‘and/or’. Optionally, the transcription factors are from a particular combination of transcription factors disclosed in Table 1, 2 or 3. In some embodiments, the transcription factor is not GLIS1, or the transcription factor is not part of a combination of transcription factors containing more than one transcription factor from the list of known reprogramming transcription factors including POU5F1, SOX2, KLF4, MYCL, NANOG, LIN28, c-MYC and GLIS1.

As used herein, a “transcriptional modulator” refers to a protein whose function is to complement the activity of transcription factors in regulating the expression of a particular gene or genes in a cell. These proteins include epigenetic modifiers that are responsible for the modification of histone acetylation and methylation and DNA methylation, and proteins involved in signaling cascades that affect gene expression, such as growth factors and protein kinases. Transcriptional modifiers can be mutant forms of proteins that are constitutively active. Examples of specific relevant transcriptional modifiers are listed in Table 4.

As used herein, a “source cell” is a cell used as the starting cell type in a cell conversion process that converts the starting cell type into a desired differentiated target cell type. The source cell is any type of cell, and can include, but is not limited to, any stem cell or differentiated cell cultured in vitro or identified in vivo as part of an organism.

As used herein, a “stem cell” is an undifferentiated or partially differentiated cell that can differentiate into various types of cells and proliferate indefinitely to produce more of the same stem cell. Stem cells include pluripotent stem cells and multipotent stem cells.

As used herein, a “pluripotent stem cell” is a cell that can differentiate into all cell types of the adult organism. Embryonic stem cells, derived from the inner cell mass of a blastocyst, an early-stage pre-implantation embryo, and induced pluripotent stem cells, generated by the expression of reprogramming transcription factors in somatic cells, are both types of pluripotent stem cell.

As used herein a “multipotent stem cell”, such as an “adult stem cell” is a multipotent cell that is able to differentiate into a small number of different cell types of a defined lineage. Examples of adult stem cells include, but are not limited to, adipose-derived stem cells, haematopoietic stem cells, mesenchymal stem cells and neural stem cells. In one alternative, the source cell according to the present invention is not an adult stem cell.

Preferably, the source cell is a differentiated cell. As used herein, a “differentiated cell” or “differentiated source cell” is any differentiated cell type defined by a specific phenotypic characteristic and may be an adult cell (such as a primary cell), or may be a cell type derived from an adult cell maintained in in vitro cell culture (such as an in vitro cultured fibroblast or cell line) or a cell derived from an adult which displays one or more detectable phenotypic characteristics of an adult organism.

As used herein, a “target cell” or “differentiated target cell” may be any differentiated cell type defined by a specific phenotypic characteristic and may be an adult cell, a progenitor cell, a partially differentiated cell, or a differentiated or partially differentiated cell type not found in adults. The term “target cell” does not encompass induced pluripotent stem cells.

As used herein the term “phenotypic characteristic” is a distinct variant or characteristic of a cell. Such characteristics can be morphological or biochemical and is an observable trait. When used in conjunction with a differentiated target cell the phenotypic trait appears in the differentiated target cell but not in the source cell. For example, the phenotypic characteristics of a dopaminergic neuron are (i) a morphology consisting of a compact cell body with long, thin axonal and dendritic protrusions, (ii) that they are electrically excitable, and (iii) express the genes LRRK2, MAP2 and PITX3. The phenotypic characteristics of an NK cell are (i) they are small, round cells that are granular in appearance, (ii) they are cytotoxic (capable of killing other cells), and (iii) express the proteins CD56 but not CD3 on their cell surface. The phenotypic characteristics of a ventricular cardiomyocyte are (i) that upon electrical stimulation, they are contractile, (ii) they have organized protein structures called sarcomeres that allow the cell to contract, and (iii) express the genes MYL2, NKX2-5 and MYH6.

As used herein, “cell conversion” refers to a method of changing a source cell into a target cell such that the phenotypic characteristics of the source cell type are changed into that of another cell type.

The term “reprogramming” refers to a method of changing a differentiated source cell into a pluripotent target cell, such as an induced pluripotent stem cell.

The term “forward programming” refers to a method of changing a pluripotent source cell, such as an embryonic stem cell or an induced pluripotent stem cell, into a differentiated target cell such that the phenotypic characteristics of the source cell type are changed into that of another cell type.

The term “transdifferentiation” refers to a method of changing a differentiated source cell into a different differentiated target cell such that the phenotypic characteristics of the source cell type are changed into that of another cell type, without transitioning through an intermediate pluripotent state.

The term “reprogramming factor-mediated transdifferentiation” refers to a method of transdifferentiation that requires the use of combinations of transcription factors taken from the list of known pluripotency inducing transcription factors, to induce the early stages of the reprogramming process in the differentiated source cell, making them more permissive to transdifferentiation. Known pluripotency inducing factor combinations include POU5F1+SOX2+KLF4+MYC; POU5F1+SOX2+NANOG+LIN28A; POU5F1+SOX2+KLF4+MYCL+LIN28A+GLIS1; and POU5F1+SOX2+KLF4+MYC/GLIS1.

The term “derived from” refers to a process whereby a first component or information from that first component, is used to isolate and make a different second component. For example, a self-replicating RNA is derived from an RNA virus wherein the RNA of the virus is engineered to remove the structural viral protein genes.

The term a “variant” in referring to a polypeptide that is at least 70%, 80%, 85%, 90%, 95%, 98%, or 99% identical to the full length polypeptide. The term “transcription factor or variants thereof” refers to the use of variants of the transcription factors described herein. The variant could be a fragment of a full-length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof and has a functional activity of interest such as the ability to promote conversion of a source cell type to a differentiated target cell type. In some aspects, the domain is at least 100, 200, 300, or 400 amino acids in length, beginning at any amino acid position in the sequence and extending toward the C-terminus. Variations known in the art to eliminate or substantially reduce the activity of the protein are preferably avoided. In some aspects, the variant lacks an N- and/or C-terminal portion of the full-length polypeptide, e.g., up to 10, 20, or 50 amino acids from either terminus is lacking. In some aspects the polypeptide has the sequence of a mature (full length) polypeptide, by which is meant a polypeptide that has had one or more portions such as a signal peptide removed during normal intracellular proteolytic processing (e.g., during co-translational or post-translational processing). In some aspects wherein the protein is produced other than by purifying it from cells that naturally express it, the protein is a chimeric polypeptide, by which is meant that it contains portions from two or more different species. In some aspects wherein a protein is produced other than by purifying it from cells that naturally express it, the protein is a derivative, by which is meant that the protein comprises additional sequences not related to the protein so long as those sequences do not substantially reduce the biological activity of the protein.

One of skill in the art will be aware of, or will readily be able to ascertain, whether a particular polypeptide variant, fragment, or derivative is functional using assays known in the art. Other convenient assays include measuring the ability to activate transcription of a reporter construct containing a transcription factor binding site operably linked to a nucleic acid sequence encoding a detectable marker such as luciferase. In certain aspects of the invention a functional variant or fragment has at least 50%, 60%, 70%, 80%, 90%, 95% or more of the activity of the full length wild type polypeptide.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous cell may be from a different organism, or it may be from the same organism. The term “exogenous” may be used interchangeably with the term “ectopic”.

The term “open reading frame” as referred to herein, is a sequence of nucleic acids that has the ability to be translated into a protein. All proteins of interest relevant to the invention, including transcription factors, are encoded as open reading frames. The term “open reading frame” may be used interchangeably with “ORF”.

The term “cell culture medium” (also referred to herein as a “culture medium” or “medium”) as referred to herein is a medium for culturing or maintaining cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, cytokines etc. Cell culture media ordinarily used for particular cell types are known to those skilled in the art.

Source cells are transfected with a self-replicating RNA to increase the expression or the amount of one or more transcription factors, or variants thereof. The term “expression” refers to the cellular processes involved in the synthesis and production of functional proteins and as appropriate, secreted proteins, including where applicable, but not limited to, for example, transcription, folding, modification and processing.

The term “self-replicating RNA”, “self-amplifying RNA”, “RNA replicon”, “self-amplifying messenger RNA”, “saRNA”, “SAM”, “sa-mRNA” and “repRNA” as used herein may be interchangeably used.

TABLE 1
Examples of one or more transcription factors involved in the cell conversion of a source differentiated cell type into the identified
differentiated target cell type (see Table 5 for Uniprot and Ensembl accession numbers for each transcription factor)

| Source cell type | Target cell type | Transcription Factors | Reference |
|---|---|---|---|
| Fibroblasts | Myocytes | MYOD1 | Lattanzi L et al. J Clin Invest. |
| | | | 1998; 101(10): 2119-2128 |
| Fibroblasts | Adipocytes | PRDM16 + CEBPB | Kajimura et al. Nature. |
| | | | 2009; 460(7259): 1154-1158 |
| Fibroblasts | Melanocytes | MITF + SOX10 + PAX3 | Yang et al. Nat Commun. 2014; 5: 5807 |
| Fibroblasts | Osteoblasts | OCT4 + OSX + L-MYC | Yamamoto et al. J Cell Biochem. |
| | | | 2016; 117(11): 2538-2545 |
| Fibroblasts | Osteoblasts | OCT4 + RUNX2 + OSX + MYCL | Yamamoto et al. Proc Natl Acad Sci USA. |
| | | | 2015; 112(19): 6152-6157 |
| Fibroblasts | Cochlea hair cell | GFI1 + POU4F3 + ATOH1 | Duran Alonso et al. PLoS One. |
| | | | 2018; 13(7): e0200210 |
| Fibroblasts | Neural crest cells | SOX10 | Kim et al. Cell Stem Cell. 2014; 15(4): 497- |
| | | | 506 |
| Fibroblasts | Neural stem cells | SOX2 | Ring et al. Cell Stem Cell. 2012; 11(1): 100- |
| | | | 109 |
| Fibroblasts | Oligodendrocytes | SOX10 + EGR2 | Mazzara et al. Nat Commun. 2017; 8: 14088 |
| Fibroblasts | Oligodendrocytes | SOX10 + OLIG2 + NKX6-2 | Chanoumidou et al. Stem Cell Reports. |
| | | | 2021; S2213-6711(21)00129-6. |
| Fibroblasts | Neurons | ASCL1 | Chanda et al. Stem Cell Reports. |
| | | | 2014; 3(2): 282-296 |
| Fibroblasts | Neurons | ASCL1 + NEUROG2 | Ladewig et al. Nat Methods. 2012; 9(6): 575- |
| | | | 578 |
| Fibroblasts | Neurons | NEUROG2 | Liu et al. Nat Commun. 2013; 4: 2183 |
| Fibroblasts | Neurons | ASCL1 + MYT1L + NEUROD2 | Yoo et al. Nature. 2011; 476(7359): 228-231 |
| Fibroblasts | Neurons | POU3F2 + MYT1L | Ambasudhan et al. Cell Stem Cell. |
| | | | 2011; 9(2): 113-118 |
| Fibroblasts | Excitatory Neurons | ASCL1 + POU3F2 + MYT1L | Torper et al. Proc Natl Acad Sci USA. |
| | | | 2013; 110(17): 7038-7043 |
| Fibroblasts | Excitatory Neurons | ASCL1 + POU3F2 + MYT1L + | Pang et al. Nature. 2011; 476(7359): 220- |
| | | NEUROD1 | 223 |
| Fibroblasts | Dopaminergic Neurons | ASCL1 + POU3F2 + MYT1L + | Pfisterer et al. Proc Natl Acad Sci USA. |
| | | LMX1A + FOXA2 | 2011; 108(25): 10343-10348 |
| Fibroblasts | Dopaminergic Neurons | ASCL1 + LMX1A + NURR1 | Caiazzo et al. Nature. 2011; 476(7359): 224- |
| | | | 227 |
| Fibroblasts | Dopaminergic Neurons | ASCL1 + NEUROG2 + SOX2 + | Liu et al. Cell Res. 2012; 22(2): 321-332 |
| | | NURR1 + PITX3 | |
| Fibroblasts | Spinal Motor Neurons | ASCL1 + POU3F2 + MYT1L + LHX3 + | Son et al. Cell Stem Cell. 2011; 9(3): 205-218 |
| | | HB9 + ISL1 + NEUROG2 + NEUROD1 | |
| Fibroblasts | Inhibitory Neurons | BCL11B + DLX1 + DLX2 | Victor et al. Neuron. 2014; 84(2): 311-323 |
| Fibroblasts | Sensory neurons | POU4F1 + NEUROG1 + NEUROG2 | Blanchard et al. Nat Neurosci. |
| | | | 2015; 18(1): 25-35 |
| Fibroblasts | Astrocytes | NFIA + NFIB + SOX9 | Caiazzo et al. Stem Cell Reports. |
| | | | 2015; 4(1): 25-36 |
| Fibroblasts | Retinal pigment | PAX6 + RAX + CRX + MITF + OTX2 + | Zhang et al. Protein Cell. 2014; 5(1): 48-58 |
| | epithelium cells | NRL + KLF4 + MYC | |
| Fibroblasts | Retinal pigment | MITF + CRX + OTX2 + LIN28 + MYCL | Woogeng et al. 2020 |
| | epithelium cells | | |
| Fibroblasts | Corneal epithelial cells | PAX6 + OVOL2 + KLF4 | Kitazawa et al. Cornea. 2019; 38 Suppl |
| | | | 1: S34-S41 |
| PBMCs | Photoreceptor cells | CRX + RAX + NEUROD1 | Komuta et al. Biol Open. 2016; 5(6): 709-719 |
| Fibroblasts | Endothelial cells | ETV2 | Morita et al. Proc Natl Acad Sci USA. |
| | | | 2015; 112(1): 160-165 |
| Amniotic cells | Endothelial cells | ETV2 + FLI1 + ERG | Ginsberg et al. Nat Protoc. |
| | | | 2015; 10(12): 1975-1985 |
| Fibroblasts | Endothelial cells | ETV2 + FLI1 + GATA2 + KLF4 | Wong et al. J Tissue Eng. |
| | | | 2016; 7: 2041731416628329 |
| Fibroblasts | Endothelial cells | ETV2 + FOXC2 + MEF2C + SOX17 + | Lee et al. Circ Res. 2017; 120(5): 848-861 |
| | | SMAD1 + HEY1 | |
| Fibroblasts | Endothelial cells | DKK3 | Chen et al. Arterioscler Thromb Vasc Biol. |
| | | | 2019; 39(4): 765-773 |
| Pancreatic | Pancreatic beta cells | PDX1 + NEUROG3 + PAX4 + MAFA | Lima et al. Diabetes. 2013; 62(8): 2821-2833 |
| exocrine cells | | | |
| Pancreatic | Pancreatic beta cells | MAPK + STAT3 | Lemper et al. Cell Death Differ. |
| exocrine cells | | | 2015; 22(7): 1117-1130 |
| Hepatocytes | Pancreatic beta cells | PDX1 + PAX4 + MAFA | Berneman-Zeitouni et al. PLoS One. |
| | | | 2014; 9(2): e87812 |
| Fibroblasts | Hepatocytes | HNF1A + HNF4A + FOXA3 | Huang et al. Cell Stem Cell. 2014; 14(3): 370- |
| | | | 384 |
| Fibroblasts | Hepatocytes | ATF5 + PROX1 + FOXA2 + FOXA3 + | Nakamori et al. Sci Rep. 2017; 7(1): 16675 |
| | | HNF4A | |
| Fibroblasts | Hepatocytes | HNF1A + HNF4A + HNF6 + CEBPA + | Du et al. Cell Stem Cell. 2014; 14(3): 394-403 |
| | | ATF5 + PROX1 + C-MYC | |
| Fibroblasts | Smooth muscle | MYOCD + MEF2C + GATA6 | Hirai et al. Arterioscler Thromb Vasc Biol. |
| | | | 2018; 38(9): 2191-2197 |
| Fibroblasts | Cardiac progenitors | ETS2 + MESP1 | Islas et al. Proc Natl Acad Sci USA. |
| | | | 2012; 109(32): 13016-13021 |
| Fibroblasts | Cardiomyocytes | GATA4 + MEF2C + TBX5 + ESRRG + | Fu et el. Stem Cell Reports. 2013; 1(3): 235- |
| | | MESP1 + MYOCD + ZFPM2 | 247 |
| Fibroblasts | Cardiomyocytes | GATA4 + HAND2 + MYOCD + TBX5 | Nam et al. Proc Natl Acad Sci USA. |
| | | | 2013; 110(14): 5588-5593 |
| Fibroblasts | Cardiomyocytes | GATA4 + MEF2C + TBX5 + MESP1 + | Wada et el. Proc Natl Acad Sci USA. |
| | | MYOCD | 2013; 110(31): 12667-12672 |
| Fibroblasts | Dendritic cells | SPI1 + IRF8 + BATF3 | Rosa et al. Sci Immunol. |
| | | | 2018; 3(30): eaau4292 |
| Fibroblasts | Macrophages | SPI1 + CEBPA | Feng et al. PNAS, 2008; 105(16), 6057- |
| | | | 6062. |
| Endothelial cells | Hepatic progenitor cells | FOXA3 + HNF1A + ONECUT1 | Inada et al. Nature Communications, |
| | | | 2020; 11(1), 1-17. |
| Endothelial cells | Haematopoietic | FOSB + GFI1 + RUNX1 + SPI1 | Sandler et al. Nature. 2014; 511(7509): 312- |
| | multipotent cells | | 318 |
| Fibroblasts | Renal tubular epithelial | EMX2 + HNF1B + HNF4A + PAX8 | Kaminski et al. Nat Cell Biol. |
| | cells | | 2016; 18(12): 1269-1280 |
| Fibroblasts | Erythroid progenitor | GATA1 + TAL1 + LMO2 + MYC | Capellera-Garcia et al. Cell Rep. |
| | cells | | 2016; 15(11): 2550-2562 |

TABLE 2
Examples of one or more transcription factors involved in the cell conversion
of a pluripotent stem cell into the identified differentiated target cell type

| Source cell type | Target cell type | Transcription Factors | Reference |
|---|---|---|---|
| Pluripotent stem cells | Myocytes | MYOD1 | Pawlowski et al. Stem Cell |
| | | | Reports. 2017; 8(4): 803-812 |
| Pluripotent stem cells | Adipocytes | PPARG2 + PRDM16 + CEBPB | Ahfeldt et al. Nat Cell Biol. |
| | | | 2012; 14(2): 209-219 |
| Pluripotent stem cells | Excitatory Neurons | NEUROD1 or ATOH1 or NEUROD2 or | Goparaju et al. Sci Rep. |
| | | NEUROG1 or NEUROG2 or NEUROG3 | 2017; 7: 42367 |
| Pluripotent stem cells | Excitatory Neurons | NEUROG2 | Zhang et al. Neuron. |
| | | | 2013; 78(5): 785-798 |
| Pluripotent stem cells | Dopaminergic Neurons | TAL1 + LMX1A + NURR1 | Theka et al. Stem Cells Transl |
| | | | Med. 2013; 2(6): 473-479 |
| Pluripotent stem cells | Astrocytes | SOX9 + NF1B | Canals et al. Nat Methods. |
| | | | 2018; 15(9): 693-696 |
| Pluripotent stem cells | Astrocytes | SOX9 + NF1A | Li et al. Stem Cell Reports. |
| | | | 2018; 11(4): 998-1008 |
| Pluripotent stem cells | Microglia | SPI1 + CEBPA | Chen et al. Stem Cell Reports. |
| | | | 2021; S2213-6711(21)00140-5 |
| Pluripotent stem cells | Oligodendrocytes | SOX10 | García-León et al. Stem Cell |
| | | | Reports. 2018; 10(2): 655-672 |
| Pluripotent stem cells | Oligodendrocytes | SOX10 + NKX6-2 + OLIG2 | Pawlowski et al. Stem Cell |
| | | | Reports. 2017; 8(4): 803-812 |
| Pluripotent stem cells | Endothelial cells | ETV2 | Wang et al. Sci Adv. |
| | | | 2020; 6(30): eaba7606 |
| Pluripotent stem cells | Endothelial cells | ETV2 + FLI1 + ERG1 | Ginsberg et al. 2012 |
| Pluripotent stem cells | Megakaryocytes | GATA1 + FLI1 + TAL1 | Moreau et al. Nat Commun. |
| | | | 2017; 8: 15076 |
| Pluripotent stem cells | Haematopoietic | GATA2 + ETV2 | Jung et al. Stem Cell Res. |
| | multipotent cells | | 2016; 17(2): 401-405 |
| Pluripotent stem cells | Haematopoietic | TAL1 + LMO2 + GATA2 + ETV2 | Lange et al. Stem Cell Res. |
| | multipotent cells | | 2016; 17(2): 401-405 |
| Pluripotent stem cells | Haematopoietic | ERG + HOXA5 + HOXA9 + HOXA10 + | Sugimura et al. Nature. |
| | multipotent cells | LCOR + RUNX1 + SPI1 | 2017; 545(7655): 432-438 |
| Pluripotent stem cells | Neutrophils | ETV2 | Brok-Volchanskaya et al. Stem |
| | | | Cell Reports. 2019; 13(6): 1099- |
| | | | 1110 |
| Pluripotent stem cells | Kidney progenitors | FIGLA + PITX2 + ASCL1 + TFAP2C | Hiratsuka et al. Sci Rep. |
| | | | 2019; 9(1): 913 |
| Pluripotent stem cells | Cardiomyocytes | GATA4 + TBX5 + NKX2-5 + SMARCD3 | Dixon et al. Mol Ther. |
| | | | 2011; 19(9): 1695-1703 |
| Pluripotent stem cells | Cardiomyocytes | GATA4 + MESP1 + SMARCD3 | Hartung et al. Stem Cells Dev. |
| | | | 2013; 22(7): 1112-1125 |

TABLE 3
Examples of cell conversion and associated transcription factors described in the applicant's
technology patents (WO2017/106932 A1 and WO2018/232459 A1 and WO2018/232458 A1)

| Source cell type | Target cell type | Transcription Factors |
|---|---|---|
| Fibroblasts | Chondrocytes | BARX1 + PITX1 + SMAD6 + FOXC1 + SIX2 + AHR + JUNB + FOSB + |
| | | SOX9 + PPRX2 + SIX1 + GDF6 + TGFB3 + RCAN1 |
| Fibroblasts | Hair follicles | ZIC1 + PRRX2 + RARB + VDR + FOXD1 + CREB3 |
| Fibroblasts | CD4+ T cells | RORA + LEF1 + JUN + FOS + BACH2 |
| Fibroblasts | CD8+ T cells | RORA + FOS + SMAD7 + JUN + RUNX3 |
| Fibroblasts | NK cells | RORA + SMAD7 + FOS + JUN + NFATC2 |
| Fibroblasts | Haematopoietic stem cells | MYB + GATA1 + GFI1 + GFI1B |
| Fibroblasts | Adipose mesenchymal stem cells | NOTCH3 + HIC1 + ID1 + ESRRA + IR1 + SIX5 + SREBF1 + SNAI2 |
| Fibroblasts | Bone marrow mesenchymal stem cells | SIX1 + ID1 + HOXA7 + FOXC2 + HOXA9 + MAFB + IRX5 |
| Fibroblasts | Oligodendrocyte precursors | NKX2-1 + ANKRD1 + FOXA2 + CDH1 + ZFP42 + IGF1 + ICAM1 + FOS |
| Fibroblasts | Skeletal muscle cells | MYOG + HIC1 + MYOD1 + FOXD1 + PITX3 + SIX2 + HOXA7 + JUNB |
| Fibroblasts | Smooth muscle cells | GATA6 + LIF + JUNB + CREB3 + MEIS1 + PBX1 |
| Fibroblasts | Cardiomyocytes | BMP10 + GATA6 + TBX5 + FHL2 + NKX2-5 + HAND2 + GATA4 + |
| | | PPARGC1A + ANKRD1 + HAND1 + S100A1 + MYOCD + FHL2 + TBX20 |
| Fibroblasts | Astrocytes | SOX2 + SOX9 + ARNT2 + E2F5 + PBX1 + SMAD1 + RUNX2 |
| Fibroblasts | Epithelial cells | FOS + DBP + HES1 + FOXA2 + ESRRA + CDH1 + FOXQ1 + PAX6 |
| Fibroblasts | Endothelial cells | SOX17 + SMAD1 + TAL1 + IRF1 + TCF7L1 + MXD4 + JUNB |
| Fibroblasts | Keratinocytes | FOXQ1 + SOX9 + MAFB + CDH1 + FOS + REL |
| Keratinocytes | Chondrocytes | BARX1 + PITX1 + SMAD6 + TGFB3 + FOXC1 + SIX2 |
| Keratinocytes | Hair follicles | RUNX1T1 + ZIC1 + PRRX1 + MSX1 + EBF1 + FOXD1 + RUNX2 |
| Keratinocytes | CD4+ T cells | RORA + LEF1 + JUN + FOS + NR3C1 |
| Keratinocytes | CD8+ T cells | RORA + FOS + SMAD7 + JUN + RUNX3 |
| Keratinocytes | NK cells | RORA + SMAD7 + FOS + JUN + NFATC2 + RUNX3 |
| Keratinocytes | Haematopoietic stem cells | MYB + GATA1 + GFI1 + GFI1B |
| Keratinocytes | Adipose mesenchymal stem cells | TWIST1 + HIC1 + ID1 + MSX1 + IRF1 + HOXB7 + SNAI2 + E2F1 |
| Keratinocytes | Bone marrow mesenchymal stem cells | SIX1 + TWIST1 + ID1 + HMOX1 + FOXC2 + HOXA7 |
| Keratinocytes | Oligodendrocyte precursors | NKX2-1 + ANKRD1 + ZFP42 + FOS + IGF1 + ICAM1 + FOXA2 + CDH1 |
| Keratinocytes | Skeletal muscle cells | MYOG + MYOD1 + RF1 + PITX3 + HOXA7 + FOXD1 + SOX8 |
| Keratinocytes | Smooth muscle cells | IRF1 + GATA6 + LIF + MEIS1 |
| Keratinocytes | Epithelial cells | NOTCH1 + HR + DBP + OTX1 + ESRRA + FOXQ1 + PAX6 + IRX5 |
| Keratinocytes | Endothelial cells | SOX17 + TAL1 + SMAD1 + IRF1 + TCF7L1 |
| Pluripotent stem cells | Chondrocytes | BARX1 + PITX1 + SMAD6 + NFKB1 + HOXA11 + FOXC1 + AHR + |
| | | SIX2 + JUNB + PRRX2 + TGFB3 + IRF1 + ZEB2 + RCAN1 + IRF1 |
| Pluripotent stem cells | Hair follicles | TWIST1 + ZIC1 + NR2F2 + PRRX1 + NFKB1 + AHR |
| Pluripotent stem cells | CD4+ T cells | RORA + LEF1 + JUN + FOS + BACH2 |
| Pluripotent stem cells | CD8+ T cells | RORA + FOS + SMAD7 + JUN |
| Pluripotent stem cells | NK cells | RORA + SMAD7 + FOS + JUN + NFATC2 |
| Pluripotent stem cells | Haematopoietic stem cells | MYB + IL1B + KLF1 + GATA1 + GFI1 + GFI1B + NFE2 |
| Pluripotent stem cells | Adipose mesenchymal stem cells | TWIST 1 + SNAI2 + IRF1 + MXD4 + NFKB1 + MSX1 + HOXB7 + ESRRA |
| Pluripotent stem cells | Bone marrow mesenchymal stem cells | IRF1 + RUNX1 + CEBPB + AHR + FOXC2 + HOXA9 |
| Pluripotent stem cells | Oligodendrocyte precursors | NKX2-1 + ANKRD1 + FOXA2 + LMO3 + FOS + IGF1 + ICAM1 + CDH1 |
| Pluripotent stem cells | Skeletal muscle cells | MYOG + IRF1 + MYOD1 + FOXD1 + NFKB1 + JUNB + HOXA7 |
| Pluripotent stem cells | Smooth muscle cells | IRF1 + NFKB1 + JUNB + FOSL2 + GATA6 + MEIS1 |
| Pluripotent stem cells | Epithelial cells | MYC + IL1B + FOS + NFKB1 + ESRRA + FOXQ1 + IRF1 + PAX6 |
| Pluripotent stem cells | Endothelial cells | SOX17 + TAL1 + SMAD1 + HOXB7 + JUNB + IRF1 + NFKB1 |
| Pluripotent stem cells | Astrocytes | IRF1 + SOX9 + ARNT2 + PAX6 + SNAI2 + SOX5 + RUNX2 |
| Pluripotent stem cells | Keratinocytes | SOX9 + NFKB1 + MYC + NR2F2 + AHR + FOSL1 + FOSL2 |
| Monocyte cells | Haematopoietic stem cells | MYB + IL1B + GATA1 + GFI1 + GFI1B |
| Cardiac fibroblast cells | Cardiomyocytes | BMP10 + GATA6 + TBX5 + ANKRD1 + HAND1 + PPARGC1A + |
| | | NKX2-5 + GATA4 |
| Pluripotent stem cells | Endothelial cells | SOX17 + TAL1 + HOXB7 + NFKB1 + IRF1 + JUNB + SMAD1 |
| Pluripotent stem cells | Astrocytes | PAX6 + POU3F2 + SNAI2 + RUNX2 + SOX5 + E2F5 + HMGB2 |
| Pluripotent stem cells | Keratinocytes | TP63 + TFAP2A + MYC + NFKBIA + SOX9 + NFKB1 |
| Bone marrow stem cells | Astrocytes | SOX2 + SOX9 + ARNT2 + MYBL2 + POU3F2 + E2F1 + HMGB2 |

TABLE 4
Examples of transcriptional modulators that are able to modulate the gene expression profile of cells

| Modulator family | Gene name of transcriptional modulator |
|---|---|
| Chromodomain proteins | CHD1, MSL3, CBX1, CBX2 |
| Bromodomain proteins | ARID1A, ARID1B, SMARCA2, SMARCA4, SMARCB1, SMARCC1, SMARCC2, SMARCD1, |
| | SMARCD2, SMARCD3, SMARCE1, PBRM1, ACTL6A, ACTL6B |
| Polycomb associated proteins | JARID2, SUZ12 |
| Histone deacetylases | HDAC1, HDAC2, HDAC3, HDAC4, HDAC5, HDAC6, HDAC7, HDAC8, HDAC9, HDAC10, |
| | HDAC11, SIRT1, SIRT2, SIRT3, SIRT4, SIRT5, SIRT6, SIRT7 |
| Histone acetyl transferases | KAT3A (CREBBP), KAT3B (EP300), KAT1, KAT2A, KAT2B, KAT5, KAT5B, KAT6A, KAT6B, |
| | KAT7, KAT8, KAT9, KAT10, KAT12 |
| Histone demethylases | KDM1A, KDM1B, KDM2A, KDM2B, KDM3A, KDM3B, JMJD1C, KDM4A, KDM4B, KDM4C, |
| | KDM4D, KDM5A, KDM5B, KDM5C, KDM5D, KDM6A, KDM6B, PHF8, UTY |
| DNA methyltransferases | DNMT1, DNMT3A, DNMT3B, DNMT3C, DNMT3L |
| Protein methyltransferase | PRMT1, PRMT4, PRMT5, PRMT6 |
| Growth factors | AM, ANGPT1, ANGPT2, ANGPT3, ANGPT4, BMP1, BMP2, BMP3, BMP4, BMP5, BMP6, |
| | BMP7, BMP8A, BMP8B, BMP10, BMP11, GDF8, GDF10, GDF5, CNTF, LIF, EGF, CSF1, |
| | CSF2, CSF3, EFNA1, EFNA2, EFNA3, EFNA4, EFNA5, EFNB1, EFNB2, EFNB3, EPO, FGF1, |
| | FGF2, FGF3, FGF4, FGF5, FGF6, FGF7, FGF8, FGF9, FGF10, FGF11, FGF12, FGF13, FGF14, |
| | FGF15, FGF16, FGF17, FGF18, FGF19, FGF20, FGF21, FGF22, FGF23, GDNF, NRTN, |
| | ARTN, PSPN, GDF9, HGF, INS, IGF1, IGF2, IL1, IL2, IL3, IL4, IL5, IL6, IL7, KGF, MSF, MSP, |
| | NRG1, NRG2, NRG3, NRG4, BDNF, NGF, NT3, NT4, PGF, PDGF, TCGF, TPO, TNF, VEGF, |
| | SHH, IDH, DHH, TGFB1, TGFB2, TGFB3, INHBA, INHBB, INHBC, INHBE, WNT1, WNT2, |
| | WNT2B, WNT3, WNT3A, WNT4, WNT5A, WNT5B, WNT6, WNT7A, WNT7B, WNT8A, |
| | WNT8B, WNT9A, WNT9B, WNT10A, WNT10B, WNT11, WNT16 |
| Signalling proteins | KRAS, MAPK |
| Mutant proteins | P53DD, KRASG12V, mutant MYC |

TABLE 5
Uniprot and Ensembl sequence accession numbers for transcription
factors detailed in Table 1 and Table 2

| Transcription Factor | UniProt ID | Ensembl ID |
|---|---|---|
| ATF5 | Q9Y2D1 | ENSG00000169136 |
| ATOH1 | Q92858 | ENSG00000172238 |
| BATF3 | Q9NR55 | ENSG00000123685 |
| BCL11B | Q9C0K0 | ENSG00000127152 |
| CEBPA | P49715 | ENSG00000245848 |
| CEBPB | P17676 | ENSG00000172216 |
| CRX | O43186 | ENSG00000105392 |
| DKK3 | Q9UBP4 | ENSG00000050165 |
| DLX1 | P56177 | ENSG00000144355 |
| DLX2 | Q07687 | ENSG00000115844 |
| EGR2 | P11161 | ENSG00000122877 |
| EMX2 | Q04743 | ENSG00000170370 |
| ERG | Q14534 | ENSG00000104549 |
| ESRRG | P62508 | ENSG00000196482 |
| ETS2 | P15036 | ENSG00000157557 |
| ETV2 | O00321 | ENSG00000105672 |
| FLI1 | Q01543 | ENSG00000151702 |
| FOSB | P53539 | ENSG00000125740 |
| FOXA2 | Q9Y261 | ENSG00000125798 |
| FOXA3 | P55318 | ENSG00000170608 |
| FOXC2 | Q99958 | ENSG00000176692 |
| GATA1 | P15976 | ENSG00000102145 |
| GATA2 | P23769 | ENSG00000179348 |
| GATA4 | P43694 | ENSG00000136574 |
| GATA6 | Q92908 | ENSG00000141448 |
| GFI1 | Q99684 | ENSG00000162676 |
| HAND2 | P61296 | ENSG00000164107 |
| HB9 | P50219 | ENSG00000130675 |
| HEY1 | Q9Y5J3 | ENSG00000164683 |
| HNF1A | P20823 | ENSG00000135100 |
| HNF1B | P35680 | ENSG00000275410 |
| HNF4A | P41235 | ENSG00000101076 |
| IRF8 | Q02556 | ENSG00000140968 |
| ISL1 | P61371 | ENSG00000016082 |
| KLF4 | O43474 | ENSG00000136826 |
| LHX3 | Q9UBR4 | ENSG00000107187 |
| LIN28 | Q9H9Z2 | ENSG00000131914 |
| LMO2 | P25791 | ENSG00000135363 |
| LMX1A | Q8TE12 | ENSG00000162761 |
| MAFA | Q8NHW3 | ENSG00000182759 |
| MEF2C | Q06413 | ENSG00000081189 |
| MESP1 | Q9BRJ9 | ENSG00000166823 |
| MITF | O75030 | ENSG00000187098 |
| MYC | P01106 | ENSG00000136997 |
| MYCL | P12524 | ENSG00000116990 |
| MYOCD | Q8IZQ8 | ENSG00000141052 |
| MYOD1 | P15172 | ENSG00000129152 |
| MYT1L | Q9UL68 | ENSG00000186487 |
| NEUROD1 | Q13562 | ENSG00000162992 |
| NEUROG1 | Q92886 | ENSG00000181965 |
| NEUROG2 | Q9H2A3 | ENSG00000178403 |
| NEUROG3 | Q9Y4Z2 | ENSG00000122859 |
| NFIA | Q12857 | ENSG00000162599 |
| NFIB | O00712 | ENSG00000147862 |
| NKX6-2 | Q9C056 | ENSG00000148826 |
| NRL | P54845 | ENSG00000129535 |
| NURR1 | P43354 | ENSG00000153234 |
| OLIG2 | Q13516 | ENSG00000205927 |
| ONECUT1 (HNF6) | Q9UBC0 | ENSG00000169856 |
| OSX | Q8TDD2 | ENSG00000170374 |
| OTX2 | P32243 | ENSG00000165588 |
| OVOL2 | Q9BRP0 | ENSG00000125850 |
| PAX3 | P23760 | ENSG00000135903 |
| PAX4 | O43316 | ENSG00000106331 |
| PAX6 | P26367 | ENSG00000007372 |
| PAX8 | Q06710 | ENSG00000125618 |
| PDX1 | P52945 | ENSG00000139515 |
| PITX3 | O75364 | ENSG00000107859 |
| POU3F2 | P20265 | ENSG00000184486 |
| POU4F1 | Q01851 | ENSG00000152192 |
| POU4F3 | Q15319 | ENSG00000091010 |
| POU5F1 | Q01860 | ENSG00000204531 |
| PRDM16 | Q9HAZ2 | ENSG00000142611 |
| PROX1 | Q92786 | ENSG00000117707 |
| RAX | Q9Y2V3 | ENSG00000134438 |
| RUNX1 | Q01196 | ENSG00000159216 |
| RUNX2 | Q13950 | ENSG00000124813 |
| SMAD1 | Q15797 | ENSG00000170365 |
| SOX10 | P56693 | ENSG00000100146 |
| SOX17 | Q9H6I2 | ENSG00000164736 |
| SOX2 | P48431 | ENSG00000181449 |
| SOX9 | P48436 | ENSG00000125398 |
| SPI1 | P17947 | ENSG00000066336 |
| STAT3 | P40763 | ENSG00000168610 |
| TAL1 | P17542 | ENSG00000162367 |
| TBX5 | Q99593 | ENSG00000089225 |
| ZFPM2 | Q8WW38 | ENSG00000169946 |

The present invention provides improved methods, and materials, to enable the cell conversion of a source cell into a cell exhibiting at least one phenotypic characteristic of a differentiated target cell. The methods involve introducing at least one self-replicating RNA encoding at least one transcription factor into a source cell, such that the at least one transcription factor is expressed, and culturing the cell to allow for cell conversion to a cell exhibiting at least one phenotypic characteristic of the differentiated target cell.

A self-replicating RNA is an RNA element derived from an RNA virus that encodes sequences and proteins that allow the RNA to transcribe and replicate itself when introduced into a host cell and allows for the amplification of expression of a desired gene product. When introduced into a mammalian cell, the self-replicating RNA molecules are translated by the host translational machinery to produce non-structural proteins of the RNA virus, which act alongside 5′- and 3′-end cis-active replication sequences to allow the RNA to replicate. Exogenous proteins can be encoded by the self-replicating RNA to achieve expression alongside the non-structural proteins of the RNA virus. The RNA virus that the self-replicating RNA is derived from is often an alphavirus, with studied examples including Sindbis Virus (SINV), Semliki Forest Virus (SFV), Eastern Equine Encephalitis Virus (EEEV), and Venezuelan Equine Encephalitis Virus (VEEV).

Self-replicating RNAs contain the basic elements of mRNA (a cap, 5′ UTR, 3′ UTR, and poly(A) tail of variable length) but have a large open reading frame at the 5′ end that encodes four viral non-structural proteins (nsP1-4) and a subgenomic promoter. Genes in the viral genome that are normally downstream of the subgenomic promoter and encode the viral structural proteins are replaced by exogenous sequence that it is desirable to express (Lundstrom 2020 International Journal of Molecular Sciences, 21(14), 1-29; doi.org/10.3390/ijms21145130). If desired, the exogenous sequence may be fused in frame to other open reading frames using self-cleaving 2A peptides in the self-replicating RNA and/or may be under the control of an internal ribosome entry site (IRES).

Subsequently, a preferred self-replicating RNA molecule encodes (i) viral non-structural proteins (e.g. nsP1-4 derived from Venezuelan Equine Encephalitis Virus) which can transcribe and replicate RNA from the self-replicating RNA molecule and (ii) a desired open reading frame encoding a transcription factor, e.g., as listed in Tables 1-3. In some embodiments the RNA may have additional (downstream or upstream) open reading frames e.g. that encode further desired gene products, which can be under the control of an IRES.

In one aspect, the at least one self-replicating RNA molecule is derived from or based on an alphavirus. In other aspects, the self-replicating RNA molecule is derived from or based on a virus other than an alphavirus, preferably, positive-stranded RNA viruses, and more preferably a picornavirus, flavivirus, rubivirus, pestivirus, hepacivirus, calicivirus, or coronavirus. Suitable wild-type alphavirus sequences are well-known. Representative examples of suitable alphaviruses include Aura, Bebaru virus, Cabassou, Chikungunya virus, Eastern equine encephalomyelitis virus, Fort Morgan, Getah virus, Kyzylagach, Mayaro, Mayaro virus, Middleburg, Mucambo virus, Ndumu, Pixuna virus, Ross River virus, Semliki Forest, Sindbis virus, Tonate, Triniti, Una, Venezuelan equine encephalomyelitis, Western equine encephalomyelitis, Whataroa, and Y-62-33. Preferably, the RNA replicon comprises or is derived from a virus selected from the group of species consisting of Venezuelan Equine Encephalitis Virus, Semliki Forest Virus and Sindbis Virus. In other aspects, the self-replicating RNA molecule is derived from or based on a virus from the list comprising: Orthomyxoviruses; Paramyxoviridae viruses; Metapneumovirus and Morbilliviruses; Pneumoviruses; Paramyxoviruses; Poxviridae; Metapneumoviruses; Morbilliviruses; Picomaviruses; Enteroviruseses; Bunyaviruses; Phlebovirus; Nairovirus; Hepamaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses; and Papovaviruses.

In one aspect, the self-replicating RNA comprises at least one promoter, either genomic or subgenomic. Preferably, the promoter is a subgenomic promoter. Preferably, the subgenomic promoter is the 26S subgenomic promoter derived from an alphavirus. Preferably, the sub genomic promoter is the 26S subgenomic promoter, which is provided herein as Sequence ID NO: 1, as follows:

(SEQUENCE ID NO: 1)
GGGCCCCTATAACTCTCTACGGCTAACCTGAATGGACTACGACAT

In one embodiment, the described promoter is operably linked to a sequence encoding at least one transcription factor of interest, e.g., as listed in one of Tables 1-3. In another embodiment, a first promoter is operably linked to a sequence encoding at least one transcription factor of interest and a second promoter is operably linked to sequence encoding another at least one transcription factor of interest.

The self-replicating RNA may further comprise a linker sequence disposed between sequences encoding at least two transcription factors of interest. In one embodiment, the linker sequence comprises a sequence that encodes a peptide spacer that is configured to be digested to thereby separate the at least two transcription factors of interest. Therefore, preferably the spacer sequence is disposed between the sequences encoding the at least two transcription factors of interest.

As such, the spacer sequence is preferably a cleavable peptide, for example a 2A peptide (WO2020/254804). Suitable 2A peptides include the porcine teschovirus-i 2A (P2A)-ATNFSLLKQAGDVEENPGP (SEQUENCE ID NO: 2), thosea asigna virus 2A (T2A)-QCTNYALLKLAGDVESNPGP (SEQUENCE ID NO: 3), equine rhinitis Avirus 2A (E2A), and Foot and mouth disease virus 2A (F2A) VKQTLNFDLLKLAGDVESNPGP (SEQUENCE ID NO: 4).

In one embodiment, the sequence encoding the at least two transcription factors of interest may be separated by a stop codon followed by an internal ribosome entry site (IRES) sequence capable of initiating translation of the downstream sequence. Typical IRES sequences include those such as the IRES sequence of encephalomyocarditis virus or vascular endothelial growth factor and type I collagen-inducible protein (VCIP), and would be known to those skilled in the Art.

Therefore, preferably the IRES sequence is disposed between the sequence encoding the at least two transcription factors of interest. Where multiple sequences encoding at least two transcription factors of interest are used, spacer sequences may include combinations of known cleavage sequences and/or IRES sequences.

In one embodiment, IRES is encoded by a nucleotide sequence as defined in SEQUENCE ID NO: 5, as follows

(Sequence ID NO: 5)
GCTAGCAATAAGGCCGGTGTGCGTTTGTCTATATGTTATTTTCCACCAT
ATTGCCGTCTTTTGGCAATGTGAGGGCCCGGAAACCTGGCCCTGTCTTC
TTGACGAGCATTCCTAGGGGTCTTTCCCCTCTCGCCAAAGGAATGCAAG
GTCTGTTGAATGTCGTGAAGGAAGCAGTTCCTCTGGAAGCTTCTTGAAG
ACAAACAACGTCTGTAGCGACCCTTTGCAGGCAGCGGAACCCCCCACCT
GGCGACAGGTGCCTCTGCGGCCAAAAGCCACGTGTATAAGATACACCTG
CAAAGGCGGCACAACCCCAGTGCCACGTTGTGAGTTGGATAGTTGTGGA
AAGAGTCAAATGGCTCTCCTCAAGCGTATTCAACAAGGGGCTGAAGGAT
GCCCAGAAGGTACCCCATTGTATGGGATCTGATCTGGGGCCTCGGTGCA
CATGCTTTACATGTGTTTAGTCGAGGTTAAAAAAACGTCTAGGCCCCCC
GAACCACGGGGACGTGGTTTTCCTTTGAAAAACACGATAAT

In another embodiment, the sequences encoding the at least two transcription factors of interest may be separated by a stop codon followed by a second subgenomic promotor sequence, which is either identical or different to the first subgenomic promoter sequence, capable of initiating transcription of the downstream sequence.

In another embodiment, the self-replicating RNA may further comprise sequence encoding proteins of interest in addition to the at least one transcription factor of interest in the described invention. These proteins of interest may be, transcriptional modulators, fluorescent reporters (e.g. GFP, mCherry, BFP, mKate, etc), antibiotic resistance genes (e.g. puromycin, blasticidin, hygromycin, neomycin, zeomycin), or inhibitors of type I and type Ill interferon immune responses (such as the vaccinia virus protein B18R). These proteins of interest may be separated from the at least one transcription factor of interest as described within.

Preferably, the self-replicating RNA comprises a poly(A) tail. Preferably, the poly(A) tail is disposed at the 3′ end of the replicon. The replicon may further comprise a 5′ cap. In the context of the present invention, the term “5′-cap” includes a 5′-cap analog that resembles the RNA cap structure and is modified to possess the ability to stabilize RNA and/or enhance translation of RNA if attached there, in a cell. The 5′ cap can be those known to persons of skill in the art, e.g. a 7-methylguanylate cap, or the anti-reverse cap analog 3′-O—Me-m7G(5′)ppp(5′)G or another analog cap structures.

In an embodiment, the self-replicating RNA comprises, preferably 5′ to 3′, a 5′ cap, a 5′ UTR, a sequence encoding at least one non-structural protein, a subgenomic promoter, a sequence encoding at least one transcription factor, a 3′ UTR and a poly(A) tail.

The self-replicating RNAs of the invention may be made using a DNA plasmid as a template. RNA copies may then be made by in vitro transcription using a polymerase, such as T7 polymerase, with the T7 promoter encoded within the DNA plasmid template 5′ of the RNA replicon sequence. Other RNA polymerases could be used instead of T7 polymerase, for example the SP6 or the T3 polymerase, in which case the DNA plasmid template of the RNA replicon may comprise the SP6 or T3 promoter instead.

In an embodiment, the self-replicating RNA of the invention may be delivered in a cassette comprising a nucleic acid sequence encoding the self-replicating RNA. For example, the self-replicating RNA may be encoded in a DNA plasmid or adenovirus or lentivirus. The DNA plasmid templates encoding the self-replicating RNA may also include other functional elements. For example, they may further comprise a variety of other functional elements including a suitable promoter for initiating transgene expression upon introduction of the vector in a host cell. For instance, the vector is preferably capable of autonomously replicating in the nucleus of the source cell. In this case, elements which induce or regulate DNA replication maybe required in the recombinant vector. Alternatively, the DNA plasmid template maybe designed such that it integrates into the genome of a host cell. In this case, DNA sequences which favour targeted integration (e.g. by homologous recombination) are envisaged.

Suitable promoters may include the SV40 promoter, CMV, EF1a, PGK, viral long terminal repeats, as well as inducible promoters, such as the Tetracycline inducible system, as examples. The DNA plasmid template may also comprise a terminator, such as the Beta globin, SV40 polyadenylation sequences or synthetic polyadenylation sequences. The DNA plasmid template may also comprise a promoter or regulator or enhancer to control expression of the nucleic acid as required. The promoter or regulator or enhancer may confer tissue specific expression of the RNA replicon on the DNA plasmid template.

Accordingly, the present invention provides in an embodiment, a method for the conversion of a source cell to a differentiated target cell by introducing at least one self-replicating RNA encoding at least one transcription factor into said source cell, such that the at least one transcription factor is expressed, and maintaining the cell to allow for cell conversion to a cell exhibiting at least one phenotypic characteristic of the target differentiated cell type.

The host cell may be a eukaryotic or prokaryotic host cell. Preferably, the host cell is a eukaryotic host cell. More preferably, the host cell is a mammalian host cell.

In the methods of the invention, the efficiency of self-replicating RNA-mediated cell conversion can be improved by inhibiting the source cell's immune response pathways in response to introduction of the self-replicating RNA. Preferably, the inhibitor of the source cell's immune response pathways, is an inhibitor of the cell's interferon response or dsRNA response pathways. For example, the efficiency of cell conversion can be improved by including recombinant vaccinia virus B18R protein, a type I interferon decoy, or by including Ruxolitinib, a JAK1/2 inhibitor. Small molecule or protein inhibitors of the interferon response pathway include, but are not limited to, fedratinib, upadacitinib, filgotinib, baricitinib, deucravacitinib, ritlecitinib, decernotinib, RIG011, BX795, MRT67307 and Y136R. In an embodiment, the inhibitor of the source cell's immune response pathways, can be an ORF encoded within a self-replicating RNA, such that it is expressed when introduced into a source cell. For example, the efficiency of cell conversion can be improved by introducing to the source cell a self-replicating RNA that encodes the vaccinia virus protein B18R, a type I interferon decoy, at the same time as the at least one self-replicating RNA encoding at least one transcription factor required for the desired cell conversion.

In an embodiment the self-replicating RNA comprises one or more modified bases. For example, the backbone can be stabilised by the introduction of one or more phosphororthioate modifications. In an alternative embodiment, 5-methylcytosine, pseudouridine and 1-methylpseudouridine. In more detail, in an embodiment the invention provides compositions comprising a self-replicating RNA encoding at least one transcription factor and comprising at least one nucleoside which has at least one chemical modification, wherein the modified nucleoside is selected from the group consisting of hypoxanthine, inosine , 8-oxo-adenine, 7-substituted derivatives thereof, dihydrouracil, pseudouracil, 2-thiouracil, 4-thiouracil, 5-aminouracil, 5-(Cl-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(Cl-C6)-alkylcytosine, 5-methylcytosine, 5-(C2-C6)-alkenylcytosine, 5-(C2-C6)alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted7-deazapurine, 7-deaza-7-substitutedpurine, 7-deaza-8-substituted purine, hydrogen(abasicresidue). The self-replicating RNA may have two or more modified nucleosides and each modification may be the same or different. In particular embodiments, the nucleosides that comprise at least one chemical modification are selected from the group consisting of, or the modified nucleotide comprises a nucleoside selected from the group consisting of, dihydrouridine, methyladenosine, methylcytidine, methylguanosine, methyluridine, methylpseudouridine, thiouridine, deoxycytodine and deoxyuridine.

In an embodiment the self-replicating RNA has two, three, four, or more modified nucleotides.

In the methods of the invention the self-replicating RNA is introduced into the source cell in vitro or in vivo. Self-replicating RNA can be introduced into cells in vitro by chemical or non-chemical-based methods or as naked RNA. Chemical methods include calcium phosphate transfection, cationic polymers, lipofection with a cationic lipid, Fugene, polyethylenimine and dendrimers. Non-chemical methods include electroporation (including nucleofection), cell squeezing, sonoporation and impalefection. It can also be introduced into cells in vivo via injection of naked RNA or RNA lipid nanoparticles intravenously or locally at targeted sites.

For in vitro applications, the self-replicating RNA is preferably formulated in a cationic lipid formulation such as Lipofectamine 3000. Cationic lipid formulation for introducing self-replicating RNA in vitro can include various formulations that are based on mixtures of N-[1-(2,3-dioleyloxy)propyl]-N,N,N trimethylammonium chloride (DOTMA), dioleoylphosphatidylethanolamine (DOPE), and 2,3-dioleyloxy-N-[2(sperminecarboxamido)ethyl]-N,N-dimethyl-1-propanaminium trifluoroacetate (DOSPA), in various ratios. This includes commercially available lipid transfection reagents, including but not limited to Lipofectamine 3000, Lipofectamine 2000, Lipofectamine MessengerMAX and TransFectin. The self-replicating RNA may be formulated in a cationic nano-emulsion. The self-replicating RNA may also be introduced into the source cell via nucleofection, electroporation or by any suitable means known in the art.

The self-replicating RNA can be delivered in vivo as naked RNA (as an aqueous solution of RNA) but, it preferred to formulate the RNA in a delivery system. For in vivo use it is preferred that the RNA is administered as part of cationic lipid emulsion, biodegradable polymeric particles or lipid nanoparticle or other materials that facilitate RNA entry into the cell and protect the RNA from nucleases. Delivery systems of nucleic acids are known in the art (Pardi et al., 2020. Current Opinion in Immunology, 65, 14-20; doi.org/10.1016/j.coi.2020.01.008).

Cationic lipids typically feature a positively charged head followed by hydrophobic tails of varying composition. Generally unsaturated, short (<30 monomer) hydrocarbon chains are associated with the highest transduction efficiencies. In aqueous formulations, these lipids form micelles with positively charged surfaces that complex with (but do not necessarily encapsulate) the RNA. The Cationic lipid DOTAP (1,2-dioleoyl-sn-glycero-3-phosphocholine) emulsified with the constituents of the MF59 (squalene span 85 and Tween 80) is preferred.

Lipid nanoparticle delivery of self-replicating RNA for vaccine use is known. Typically the lipid nanoparticle is made of four components a cationic or lonisable lipid; cholesterol; a helper phospholipid and a polyethylene glycol (PEG) lipid. lonisable lipid are preferred as they allow RNA to be encapsulated in acidic conditions and at physiological pH, but as the formulation enters the cell the lipid ionizes and the RNA is allowed to separate from the particle.

In certain embodiments the self-replicating RNA may be delivered to the source cell as two molecules each containing cis active sequences allowing replication, the first carrying the non-structural genes and the second encoding the at least one transcription factor of interest.

Accordingly in one embodiment of the invention there is provided a pharmaceutical composition comprising a self-replicating RNA encoding a transcription factor and a delivery system selected from the group: lipid nanoparticles, cationic lipid emulsions and biodegradable polymeric particles.

Accordingly, the present invention described herein provides compositions of self-replicating RNA encoding at least one transcription factor, and optionally at least one other open reading frame sequence such as those encoding for transcriptional modulators, antibiotic resistance, inhibitors of type I and type III interferon responses, or reporter proteins and a delivery system to achieve a desirable cell conversion both in vitro and in vivo.

The present invention further provides a method of generating at least one cell exhibiting at least one phenotypic characteristic of a differentiated target cell from a source cell, the method comprising: increasing the amount of one or more transcription factors in the source cell by the introduction of a self-replicating RNA encoding a transcription factor, such that the at least one transcription factor is expressed, and culturing the source cell for a sufficient time and under conditions to allow cell conversion to the differentiated target cell, thereby generating the cell exhibiting at least one phenotypic characteristic of the differentiated target cell from the source cell. In one embodiment, the method comprises increasing the amount of two or more, three or more, four or more, five or more, or six or more transcription factors in the source cell by the introduction of a self-replicating RNA.

The present invention provides a method of generating at least one cell exhibiting at least one phenotypic characteristic of a differentiated target cell from a population of source cells, the method comprising: increasing the amount of one or more transcription factors by the introduction of a self-replicating RNA encoding a transcription factor in at least one cell in the population of source cells; and culturing the population of source cells for a sufficient time and under conditions to allow cell conversion to the differentiated target cell, thereby generating the population of cells exhibiting at least one phenotypic characteristic of the differentiated target cell from the population of source cells.

A source cell or a population of source cells may be any pluripotent cell or differentiated cell type described herein, including an induced pluripotent stem cell, or a differentiated cell. In some aspects, a source cell population can be a mix of different source cells. The differentiated cell may be an adult cell or a cell derived from an adult which displays one or more detectable phenotypic characteristics of an adult or non-embryonic cell. A source cell or a population of source cells of the present invention are typically mammalian cells, such as, human cells, primate cells, rodent cells (e.g. mouse or rat) equine cells and bovine cells, preferably human cells. The differentiated source cell or differentiated target cell according to the present invention may be any cell of the three embryonic germ layers, i.e., endoderm, mesoderm, and ectoderm. Examples of differentiated cells includes cardiac cells, skeletal muscle cells, smooth muscle cells, red blood cells, lung cells, pancreatic cells, neuron cells, chondrocytes, T cells, NK cells, macrophages, dendritic cells, epithelial cell or myoblast cells. A ‘differentiated cell’ as used herein may be a primary cell (non-immortalised cell) or may be a cell derived from a cell line (immortalised cell). A differentiated cell may be a healthy cell or a diseased cell.

In some aspects, the source cells can be pluripotent stem cells. In some aspects, the source cell can be an induced pluripotent stem cell. In some aspects, the source cell can be an embryonic stem cell.

In some aspects, the source cells can be multipotent stem cells. In some aspects, the source cell can be a mesenchymal stem cell. In some aspects, the source cell can be a neural stem cell.

In some aspects, the source cell can be a fibroblast. In some aspects, the source cell can be a dermal fibroblast. In some aspects, the source cell can be a gingival (oral) fibroblast. In some aspects, the source cell can be a foreskin fibroblast.

In some aspects, the source cell can be an endothelial cell. In some aspects, the source cell can be a vascular endothelial cell. In some aspects, the source cell can be a lymphatic endothelial cell.

In some aspects, the source cells can be derived from blood. In some aspects, the source cells can be isolated from peripheral blood mononuclear cells (PBMCs). In some aspects, the source cell is a blood-derived CD4+ T cell. In some aspects, the source cell is a blood-derived CD8+ T cell. In some aspects, the source cell is a blood-derived CD56+ NK cell. In some aspects, the source cell is a blood-derived CD19+ B cell.

The present invention provides a method of generating a population of differentiated target cells from a population of source cells, the method comprising: increasing the amount of one or more transcription factors in the population of source cells by introducing a self-replicating RNA encoding at least one transcription factor; and maintaining the population of source cells for a sufficient time and under conditions to allow cell conversion to the population of differentiated target cells wherein at least 0.1% of cells in the population of differentiated target cells exhibit at least one phenotypic characteristic of the differentiated target cell; thereby generating the population of differentiated target cells from the population of source cells.

In some aspects of the method of the invention, at least 0.1%, at least 0.5%, at least 1%, at least 5% or at least 10% of the differentiated target cell or population of differentiated target cells exhibit at least one phenotypic characteristic of the differentiated target cell.

In other aspects, at least 10% to 20%, preferably over 50%, more preferably over 70%, more preferably at least 85%, 95% or 100% of the differentiated target cell or population of differentiated target cells exhibit at least one phenotypic characteristic of the differentiated target cell.

In some aspects, the differentiated target cell is a neuron, macrophage, hepatocyte or muscle cell.

The methods of the invention increases the amount of one or more transcription proteins by 5%, 10%, 20%, 30%, 40%, 50%, 60%, 70%, 80%, 90%, or more relative to the original unmodified source cell.

In some aspects, at least 1% of the cells in the population of differentiated target cells exhibit at least one phenotypic characteristic of a neuron. In other embodiments the population of differentiated target cells exhibit at least one phenotypic characteristic of a muscle cell. Typically, conditions suitable for cell conversion include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days.

In any method described herein, the method may further include the step of expanding the cells exhibiting at least one phenotypic characteristic of the differentiated target cell to increase the proportion of cells in a population exhibiting that phenotypic characteristic. The step of expanding the cells may be in culture for a sufficient time and under conditions for generating a population of cells as described herein. In any method described herein, the method may further include the step of administering the cells, or cell population including a cell, exhibiting at least one phenotypic characteristic of a differentiated target cell.

The cell conversion method described herein can be utilised for autologous therapy (i.e. source cells are isolated from a subject, undergo cell conversion using self-replicating RNA as described herein, to generate differentiated target cells and then injected back into the same subject) or allogeneic therapy (i.e. source cells are isolated from a subject, undergo cell conversion using self-replicating RNA as described herein, to generate differentiated target cells and then injected back into a different subject). The method for cell conversion of source cells can be an in vivo cell conversion method, whereby the self-replicating RNA encoding at least one transcription factor is delivered to subjects and the cell conversion of the source cell to a desired differentiated target cell occurs in vivo.

### EXAMPLES

### Example 1: In vitro Transcription of Self-replicating RNA

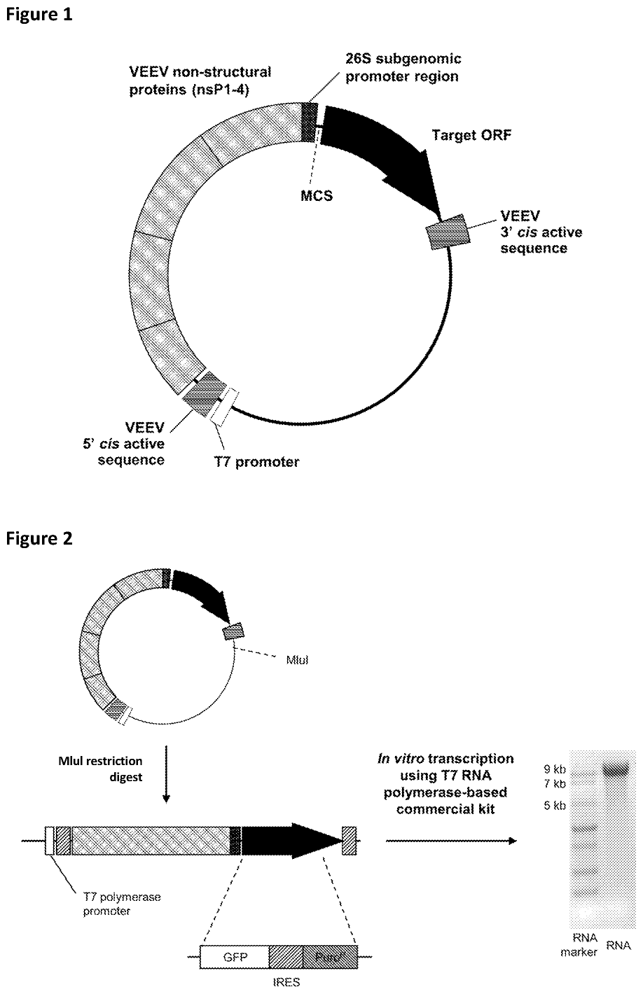

Plasmid DNA containing an alphavirus-based replicon was used as a template to generate self-replicating RNA (repRNA) by in vitro transcription. The alphavirus-based replicon utilised was derived from the Venezuelan Equine Encephalitis Virus strain TC-83 genome. It directly encodes sequence from the VEEV genome but has the structural protein coding sequences of the virus removed to prevent the production of infectious viral particles (FIG. 1). The viral non-structural protein coding region is flanked by viral 5′- and 3′-cis active sequences, a T7 promoter at the 5′- end and is followed by a synthetic polyadenylation sequence. The plasmid template was modified to include a multiple cloning site within the viral genomic sequence, immediately after the viral 26S subgenomic promoter, to allow expression of target open reading frames.

Template plasmids were linearised by Mlul restriction endonuclease digestion (FIG. 2). Linear templates were used to generate run-off transcripts by in vitro transcription employing a T7 DNA-dependent RNA polymerase. Fully capped and poly-adenylated repRNA transcripts were produced in a co-transcriptional capping reaction using the RiboMAX Large Scale RNA Production System (Promega) and the CleanCap AU Cap 1 capping reagent (TriLink Biotechnologies). In vitro transcription was performed at 37° C. for 2 hr, with 0.5 μg linearised plasmid template, with nucleotide triphosphates and CleanCap AU capping reagent at 7.5 mM final concentration. Following in vitro transcription, the plasmid template was removed by DNasel treatment at 37° C. for 15 min. RNA was precipitated on ice with 2.5M ammonium acetate, washed with 70% ethanol and reconstituted in water. RNA was analysed by measuring absorbance at 260 nm to assess purity and concentration, and by agarose gel electrophoresis to further assess purity and size (FIG. 2). The agarose gel image in FIG. 2 shows successful generation of an 11 kb in vitro transcribed mRNA, with an open reading frame encoding GFP and puromycin n-acetyltransferase (Puro^{R}), separated by an internal ribosome entry site (IRES) sequence.

### Example 2: Delivery of GFP-Expressing Self-replicating RNA and Assessment of GFP Expression in iPSC

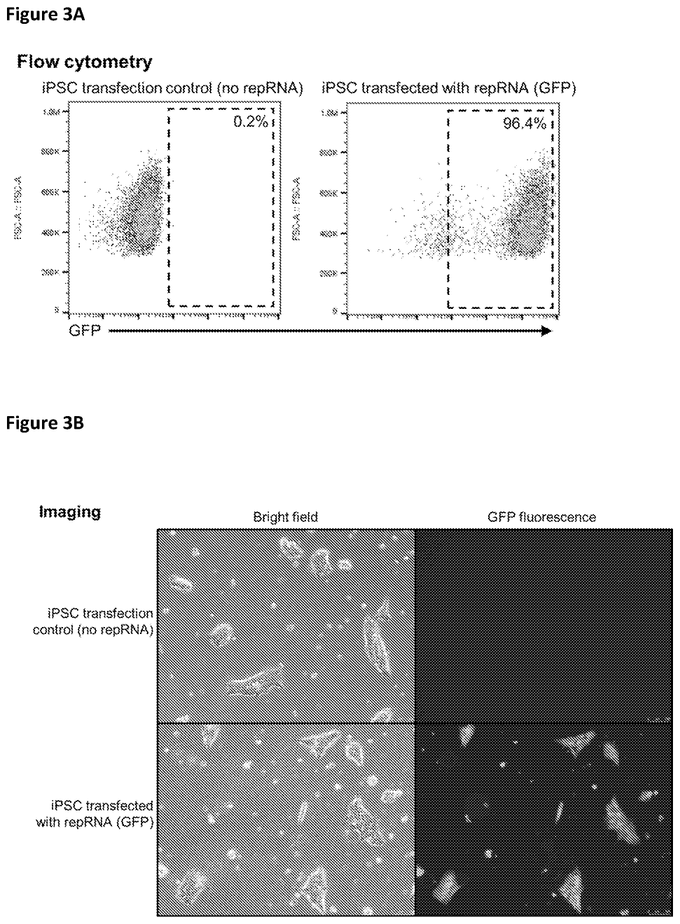

A plasmid template was constructed, with sequence inserted at the multiple cloning site of the self-replicating RNA vector, encoding GFP and puromycin n-acetyltransferase (Puro^{R}), separated by an IRES sequence (FIG. 2). This plasmid was used as a template to generate GFP-IRES-Puro^{R} repRNA by in vitro transcription (FIG. 2 shows this RNA species analysed on an agarose gel). Induced pluripotent stem cells (iPSCs) were routinely maintained on Vitronectin XF (STEMCELL Technologies) in complete E8 growth media (Thermo Fisher), passaged every 4-5 days by colony dissociation with ReLeSR (STEMCELL Technologies). Confluent cultures of iPSCs were pre-treated with 10 μM Y-27632 (STEMCELL Technologies) for 90 min prior to nucleofection. For nucleofection, iPSCs were treated for 4 min with TrypLE (Thermo Fisher) to make a single cell suspension, collected and counted. Required numbers of cells were centrifuged for 4 min at 300 g and resuspended in complete P3 buffer (P3 Primary Cell Nucleofector Kit; Lonza). iPSCs were nucleofected using the Lonza 4D system with 2 μg GFP-IRES-Puro^{R} repRNA per 1×106 cells. Nucleofected cells were plated on Vitronectin XF coated culture plates in complete E8 growth media with 10 μM Y-27632, at a density of 1×105 cm⁻². In certain experiments, puromycin (Sigma) was added to cells at a final concentration of 500 ng/mL and recombinant B18R protein (BioTechne) at a final concentration of 200 ng/ml. GFP expression was assessed by microscopy and flow cytometry (FIG. 3). Live cell fluorescent microscopy was performed on a Leica DMi1 Inverted Microscope using a GFP filter set. For flow cytometry, cells were incubated with TrypLE for 5 min and collected in PBS+1% BSA. Cells were then centrifuged for 4 min at 300g and resuspended in PBS +1% BSA +0.1 μg/mL DAPI for analysis. Prepared cells were analysed on an Attune N×T Flow Cytometer using a standard filter set (Thermo Fisher).

The data shows that the GFP-IRES-Puro^{R} repRNA was effectively expressed in iPSC, as confirmed by GFP expression observed by both flow cytometry (FIG. 3A) and microscopy (FIG. 3B). Flow cytometry analysis showed that iPSCs were transfected at extremely high efficiency with the GFP-IRES-Puro^{R} repRNA, as evidenced by 96.4% of iPSC being GFP positive.

### Example 3: Expression Kinetics of GFP Encoded by Either Self-replicating RNA, Standard Non-replicating mRNA, Plasmid DNA or Lentivirus in iPSC

A template for in vitro transcription of standard mRNA was derived by PCR from the GFP-IRES-Puro^{R} repRNA plasmid template. PCR was performed using Q5® High-Fidelity DNA Polymerase (New England Biolabs) with 1 ng of the GFP-IRES-Puro^{R} repRNA plasmid DNA as template, and with primers (Sequence ID NO: 6 and 7) designed to incorporate a T7 promoter, a 5′UTR and a canonical Kozak sequence upstream of the GFP start codon, and a 120 residue polyadenylation sequence downstream of the GFP stop codon.

| | |
|---|---|
| GFP mRNA | GCTAATACGACTCACTATAAGGACTCACTATTTGTTTTCGCGCCCAGTTGCAAAAAGTGT |
| template Primer 1 | CGCCACCATGgtgagcaagggcgaggag (Sequence ID NO: 6) |
| GFP mRNA | ttttttttttttttttttttttttttttttttttttttttttttttttttttttttttttttttttttttttt |
| template Primer 2 | tttttttttttttttttttttttttttttttttttttttttttttttTTACTTGTACAGCTCGTCCATGCC |
| | (Sequence ID NO: 7) |

The PCR product was purified using a GeneJET Gel Extraction Kit (Thermo Fisher) and quantified. Fully capped and poly-adenylated mRNA transcripts were produced in a co-transcriptional capping reaction using the HiScribe T7 High Yield RNA Synthesis Kit (New England Biolabs) and the CleanCap AG Cap 1 capping reagent (TriLink Biotechnologies). In vitro transcription was performed at 37° C. for 2 hr, with 0.5 μg PCR product template, with nucleotide triphosphates and CleanCap AG capping reagent at 5 mM and 4 mM final concentration, respectively. In the in vitro transcription reactions, UTP was fully substituted for the pseudo base N1-methyl-pseudouridine (TriLink Biotechnologies). RNA was purified using the Monarch RNA Cleanup Kit (New England Biolabs).

For generating GFP expressing lentivirus particles, Lenti-X 293T (Takara Bio) were maintained in DMEM high glucose, supplemented with 10% foetal calf serum and sodium pyruvate. Following seeding, Lenti-X 293T were transfected using Lipofectamine 2000, with psPAX2 packaging plasmid, pMD2.G envelope plasmid and a transfer plasmid encoding the EF1a promoter driving expression of GFP. Media was carefully replaced 18 hr after transfection, and virus containing supernatant was harvested at 48 hr post-transfection, before virus concentration using PEG 6000 (Sigma). Concentrated virus was diluted in DMEM and stored at −80° C. prior to use.

iPSC were seeded at 6.5×104 cm⁻² in complete E8 growth media supplemented with 10 UM Y-27632 and 6 hr later transduced with lentivirus at an MOI of 2 with addition of Polybrene to a final concentration of 6.25 μg/mL. iPSC were nucleofected with either GFP-IRES-Puro^{R} repRNA, GFP mRNA or an EF1a promoter-GFP plasmid. Transduced and nucleofected cells were maintained in culture through multiple passages and analysed by flow cytometry at set time points. A subset of GFP-IRES-Puro^{R} repRNA nucleofected iPSC were maintained in standard growth media with the addition of 500 ng/ml puromycin (from 24 hour post-nucleofection).

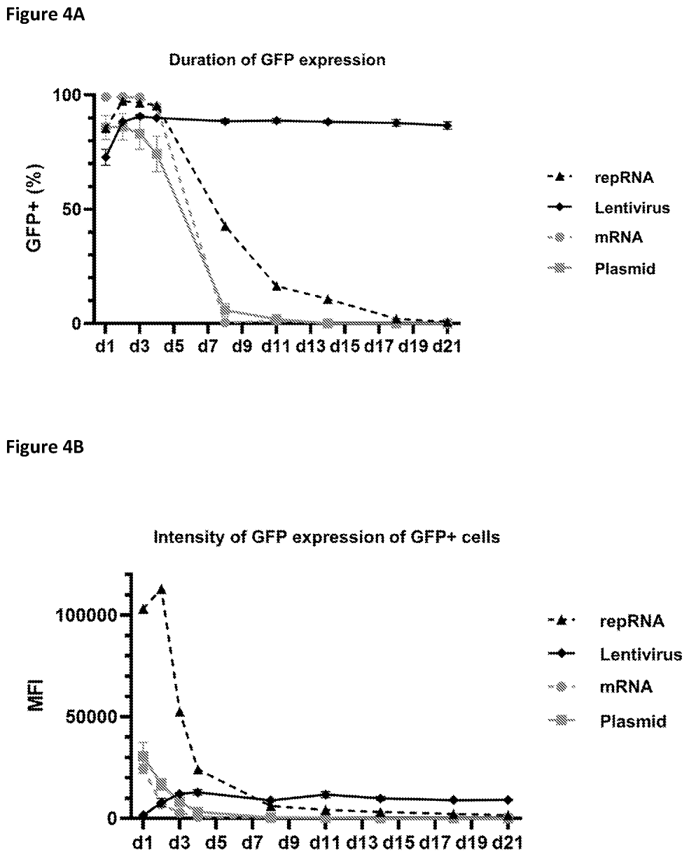

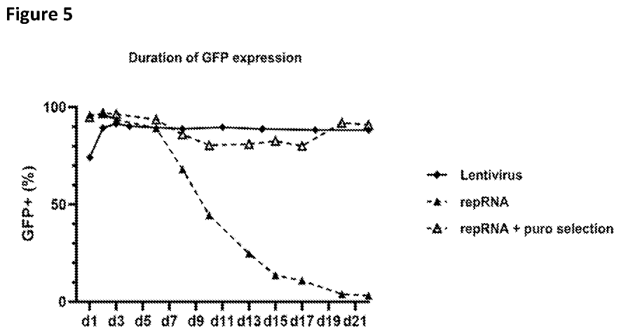

The data shows that both percentage GFP positive cells (FIG. 4A) and intensity of GFP expression (FIG. 4B) mediated by repRNA is elevated compared to GFP expression from standard mRNA and plasmid DNA at comparable time points. Lentivirus transduced cells show prolonged high percentage of GFP cells (FIG. 4A), whereas repRNA gives a far higher intensity of GFP expression in the first 4 days post transduction/nucleofection (FIG. 4B). Further, testing the functionality of the IRES-Puro^{R} cassette by applying puromycin selection showed that repRNA-mediated GFP expression can be maintained in growing iPSC cultures, with GFP expression observed in at least 80% of iPSC up to 3 weeks post-nucleofection, similar to levels observed in lentivirus transduced iPSC cultures (FIG. 5).

Together these data demonstrate that repRNA can be delivered to cells at equivalent efficiency to lentivirus, and expression from repRNA can be maintained in cells for prolonged durations without genetic modification of the host cell, unlike lentivirus, which requires integration into the host genome for its expression. Further, expression levels from repRNA are significantly higher than those achieved for any other delivery method in the first 4 days post-transfection. This represents an advantage of repRNA over current technologies for exogenous gene expression, giving increased expression levels over plasmid and mRNA, whilst obviating the genetic modification of host cells, required by lentivirus for expression.

### Example 4: Delivery of GFP-expressing Self-replicating RNA and Assessment of GFP Expression in Dermal Fibroblasts and HEK293T Cells

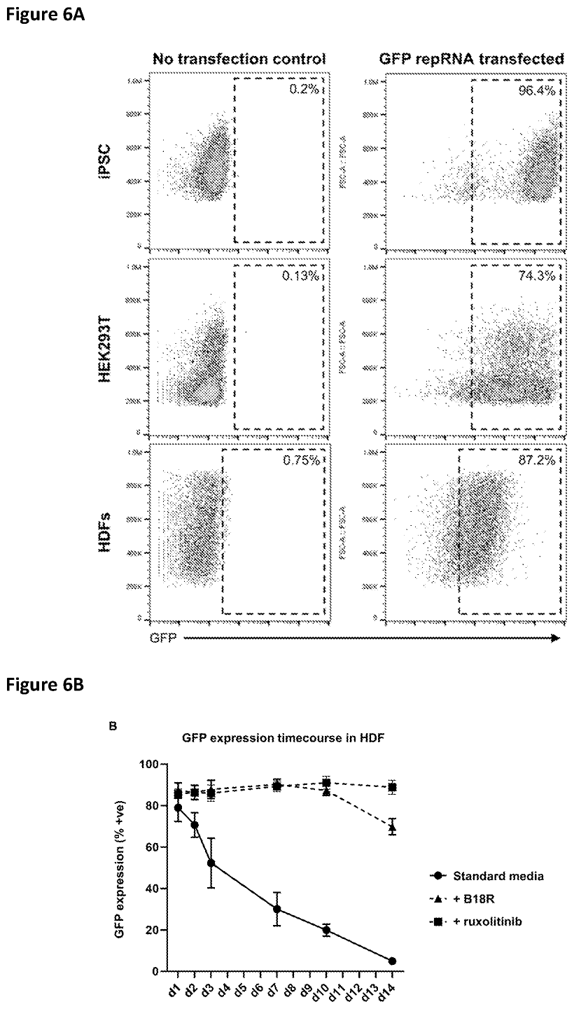

Human dermal fibroblasts (HDFs; ATCC) and HEK293T cells (ATCC) were routinely maintained in DMEM supplemented with 10% foetal calf serum. For transfection, HDFs and HEK293Ts were treated for 5 min with TrypLE to make a single cell suspension, collected and counted. Required numbers of cells were centrifuged for 4 min at 300 g and resuspended in buffer R (Neon Transfection Kit; Thermo Fisher). Cells were electroporated using the Neon Electroporation system with 1 μg GFP-IRES-Puro^{R} repRNA per 3×105 cells. Electroporated HDFs were plated at a density of 3×104 cm⁻² and HEK293T at a density of 1×105 cm⁻². GFP expression was assessed by flow cytometry at 72 hour post-transfection (FIG. 6A).

The data indicates that, as well as iPSC, standard cell lines (HEK293T) and primary cells (human dermal fibroblasts) can also be efficiently transfected and functionally express repRNA (FIG. 6A).

It has previously been demonstrated that vaccinia virus-derived B18R protein and the small molecule JAK1/2 inhibitor Ruxolitinib are able to improve the expression dynamics of self-replicating RNA, by inhibiting cellular antiviral responses (Blakney et al., 2021). In some experiments, recombinant viral B18R protein (BioTechne) or ruxolitinib (Selleckchem) were included in maintenance media at the point of transfection and through the time course of the experiment in an attempt to maintain the expression of GFP from GFP-IRES-Puro^{R} repRNA repRNA. Following transfection, HDFs were maintained in standard media, or with standard media containing 200 ng/ml B18R or 10 μM ruxolitinib, with media changes every 2-3 days, and GFP expression was assessed by flow cytometry over a time course of 10 days (FIG. 6B).

The data indicates that addition of the interferon response pathway blockers B18R or ruxolitinib to standard culture media results in maintained repRNA-mediated expression of GFP in vitro, over a time course of 14 days (FIG. 6B).

### Example 5: Conversion of iPSC to Neurons Using NEUROG2-expressing Self-replicating RNA

Rapid conversion of iPSC to neurons can be achieved efficiently by a single transcription factor and represents an ideal model system for testing cell conversions. There are multiple published reports demonstrating that forced expression of NEUROG2 is sufficient to convert iPSC to neurons in less than 7 days (Zhang et al., 2013 Neuron, 78(5), 785-798. https://doi.org/10.1016/j.neuron.2013.05.029). Following NEUROG2 expression, iPSC rapidly adopt a classic neuronal morphology, with dendrite-like projections and a small cell body. The converted cells also express NCAM at their cell surface, and neuron specific genes TUBB3, MAP2 and POU3F2 (BRN2).

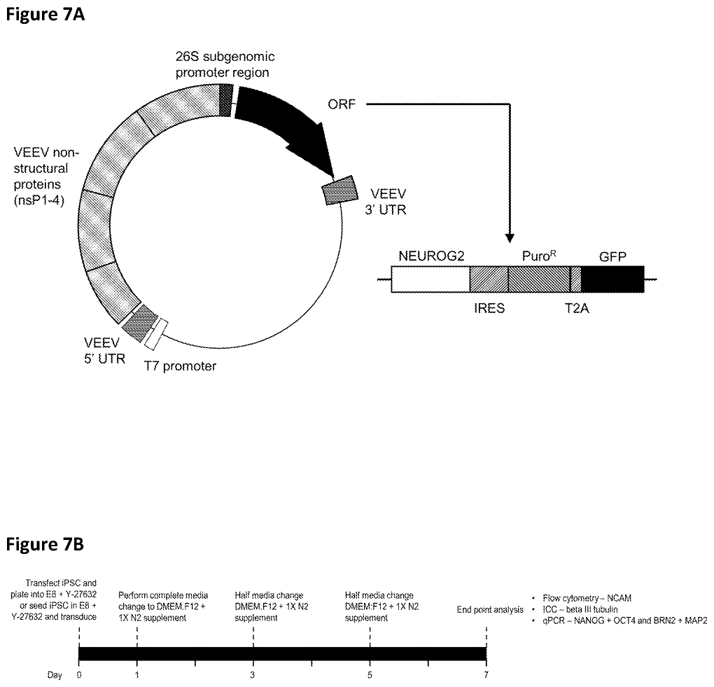

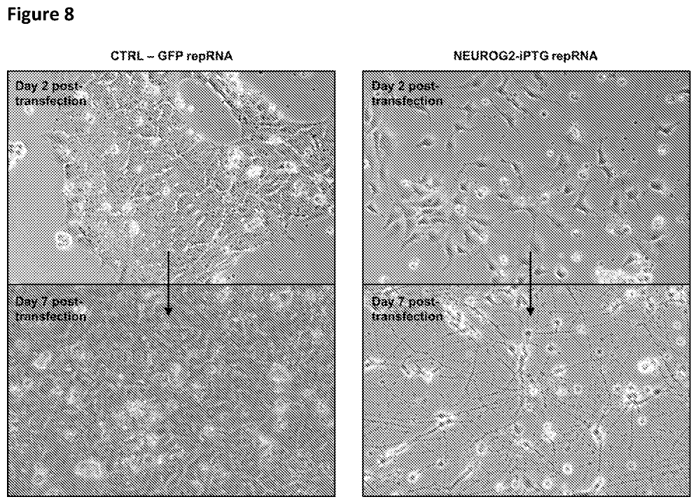

To test the ability of repRNA to promote NEUROG2 mediated cell conversion, a plasmid template was constructed, with sequence inserted at the multiple cloning site of the self-replicating RNA vector, encoding NEUROG2 and Puro^{R}-T2A-GFP separated by an IRES sequence (FIG. 7A). This plasmid was used as a template to generate NEUROG2-IRES-Puro^{R}:T2A:GFP (NEUROG2-iPTG) repRNA by in vitro transcription. For iPSC to neuron conversion experiments, iPSC were nucleofected with NEUROG2-iPTG repRNA, as previously described, and plated onto Geltrex (Thermo Fisher) coated culture plates. Media was changed 24 hours post-nucleofection, to DMEM:F12+N2 supplement (Thermo Fisher) with half media changes every 2-3 days (FIG. 7B).

Imaging showed the morphology of iPSC-derived cells at day 2 and day 7 post-nucleofection with either a control GFP-IRES-Puro^{R} repRNA or the NEUROG2-iPTG repRNA (FIG. 8). iPSC nucleofected with GFP-IRES-Puro^{R} repRNA showed typical iPSC-like morphology at both day 2 and day 7. At day 2, the NEUROG2-iPTG repRNA nucleofected cells showed a clear change in their morphology compared to the control, and by day 7 showed neuronal morphology with multiple dendrite-like extensions and a small cell body.

### Gene Expression Analysis by Quantitative PCR (qPCR)

RNA was isolated from cultured cells using the PicoPure RNA Isolation Kit (Thermo Fisher). Cells were collected by treating for 4 min with TrypLE followed by centrifugation for 4 min at 300 g, and RNA was isolated following the manufacturers protocol, with an on-column DNase digest to remove contaminant DNA. cDNA was generated from purified RNA using a SensiFAST cDNA synthesis kit (Bioline) with 1000 ng template RNA following the manufacturers protocol. Quantitative PCR was performed with pre-designed TaqMan assays (Thermo Fisher) using TaqMan Fast Advanced Master Mix (Thermo Fisher), run on a QuantStudio 5 Real Time PCR System (Thermo Fisher). FBXL12 and SRP72 were used as housekeeping gene controls. Relative gene expression was calculated using the ΔΔCT method.

### Flow Cytometry for NCAM

For flow cytometry, cells were incubated with TrypLE for 5 min and collected in PBS+1% BSA. Cells were then centrifuged for 4 min at 300 g and resuspended in PBS+1% BSA and Alexa Fluor® 647-conjugated anti-CD56 (NCAM) mouse monoclonal antibody (1:500; BioLegend) and incubated at 4° C. for 30 min. Cells were washed and resuspended in PBS+1% BSA+0.1 μg/mL DAPI. Prepared cells were analysed on an Attune NxT Flow Cytometer using a standard filter set.

### Immunocytochemistry (ICC) Detection of TUBB3

Cells were washed with PBS and fixed for 15 min at room temperature with PBS+4% paraformaldehyde. Following fixation, cells were washed with PBS, then blocked and permeabilised in one step with PBS+4% normal goat serum+0.1% Triton X-100 for 15 min at room temperature. Cells were incubated for 1 hour at room temperature with purified anti-tubulin B3 (TUBB3) antibody (1:100; BioLegend) in PBS+4% normal goat serum, and then 1 hour at room temperature with Alexa Fluor® 647-conjugated secondary antibody (1:1000; Thermo Fisher) in PBS+4% normal goat serum. Following antibody steps, cells were stored in PBS+1% BSA at 4° C. before imaging on a Leica DMi1 Inverted Microscope.

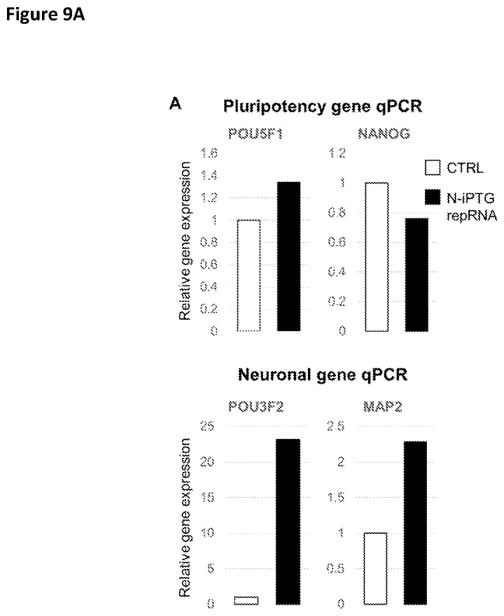

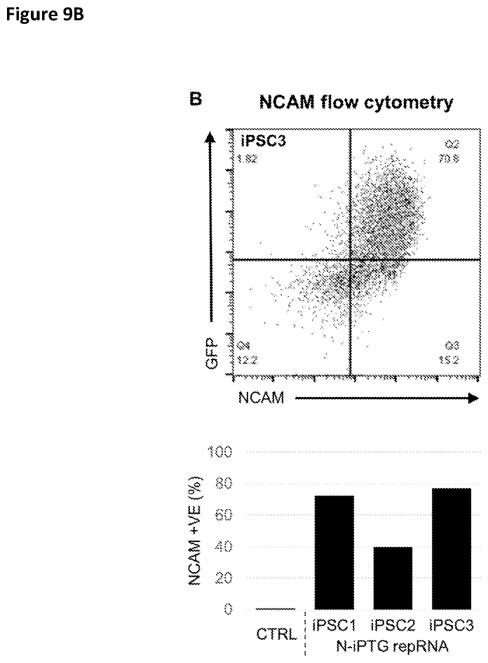

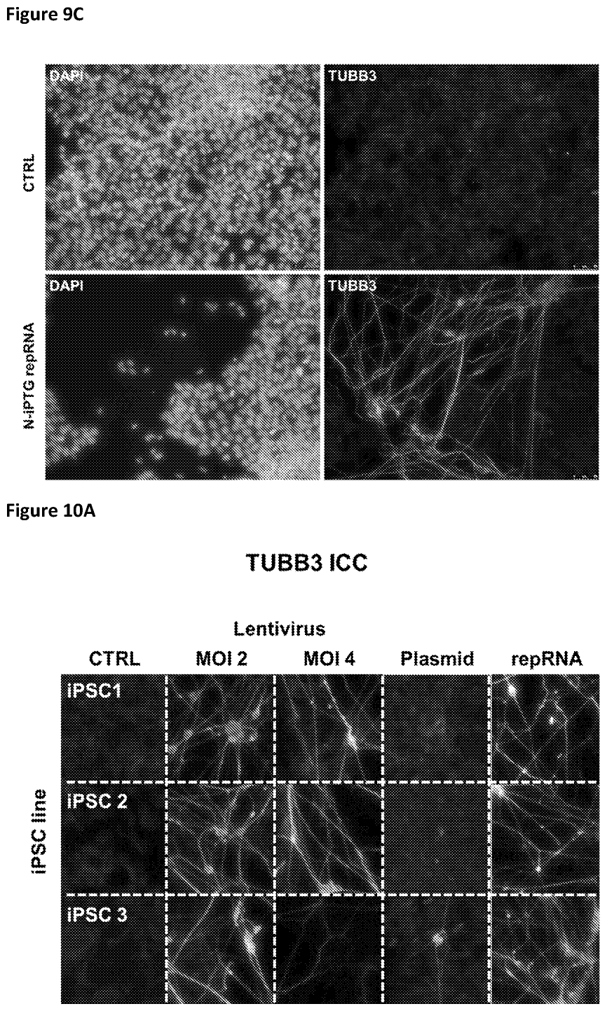

Analysis of differentiated cultures showed NEUROG2-iPTG repRNA efficiently converted iPSC to neuronal cells. Assessment of neuronal marker gene expression by qPCR at day 7 post transfection, shows an increase in neuronal specific gene expression in iPSC nucleofected with NEUROG2-iPTG repRNA, compared to control cells (FIG. 9A). Expression of pluripotency-associated genes is maintained due to presence of proliferative iPSC that were not transfected with the NEUROG2-iPTG repRNA (around 3% of initial cell population) and did not convert to neurons. Flow cytometry to detect the neuronal cell surface marker NCAM, shows a high percentage of NCAM positive cells in NEUROG2-iPTG repRNA nucleofected cultures, and further, GFP expression from NEUROG2-iPTG is concomitant with NCAM expression (FIG. 9B). Finally, ICC analysis of TUBB3 expression detects neuronal cells with expected class III β-tubulin cellular distribution in NEUROG2-iPTG repRNA nucleofected cultures, but not in control cultures (FIG. 9C).

Together these data demonstrate that repRNA-encoded NEUROG2 can be delivered to iPSC, inducing their rapid and high efficiency conversion to neurons. This illustrates that repRNA is a valid modality to deliver transcription factors for driving cell conversions.

### Example 6: Comparison of Different Methods for Delivering NEUROG2 to iPSC for Conversion to Neurons

To test the efficiency of different methods to deliver NEUROG2 to iPSC and drive their conversion to neurons, three different iPSC lines were nucleofected with either NEUROG2-iPTG repRNA or an EF1a promoter-NEUROG2 plasmid, or transduced with NEUROG2 expressing lentivirus particles. NEUROG2 expressing lentivirus particles were generated as previously described, using a transfer plasmid encoding the EF1a promoter driving expression of NEUROG2. Post-nucleofection and post-transduction, cells were maintained and analysed by ICC and flow cytometry as previously described.

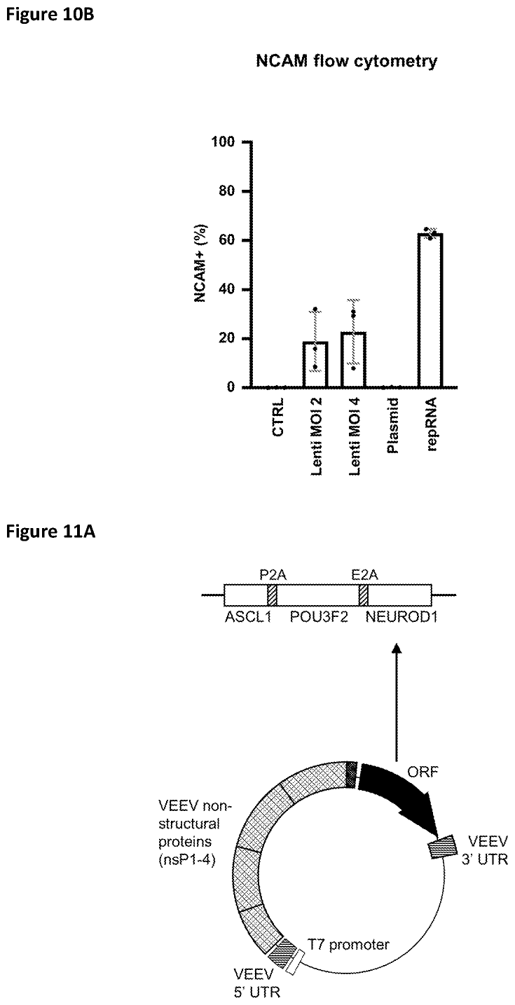

Analysis of differentiated cultures showed NEUROG2-iPTG repRNA converted iPSC from all three iPSC lines to neuronal cells at a higher efficiency than plasmid DNA or lentivirus. ICC analysis of TUBB3 expression detects neuronal cells with expected class III B-tubulin cellular distribution in cultures of iPSC converted to neurons by either NEUROG2 lentivirus or NEUROG2-iPTG repRNA nucleofected cultures (FIG. 10A). Flow cytometry to detect the neuronal cell surface marker NCAM, shows a higher percentage of NCAM positive cells in NEUROG2-iPTG repRNA nucleofected cultures than in lentivirus transduced cultures, confirmed in multiple iPSC lines (FIG. 10B).

Together these data demonstrate that delivering transcription factors by repRNA is more efficient for cell conversion than plasmid or lentivirus-mediated delivery, with the advantage of not genetically modifying the host cell in the process. The data also highlights the robustness of the protocol, as evidenced by high efficiency neuronal conversion of cells from three different iPSC lines from different donors.

### Example 7: Conversion of Dermal Fibroblasts to Neurons Using ASCL1, POU3F2 (BRN2), NEUROD1 and MYT1L-expressing Self-replicating RNA

Conversion of fibroblasts to neurons can be achieved by expression of the transcription factors ASCL1, POU3F2 (BRN2), NEUROD1 and MYT1L in dermal fibroblasts (Pang et al., 2011 Nature, 476(7359), 220-223; doi.org/10.1038/nature10202). To further exemplify the utility of self-replicating RNA for cell conversions, ASCL1, POU3F2, NEUROD1 and MYT1L expressing self-replicating RNA were tested for their ability to convert HDFs to neurons.

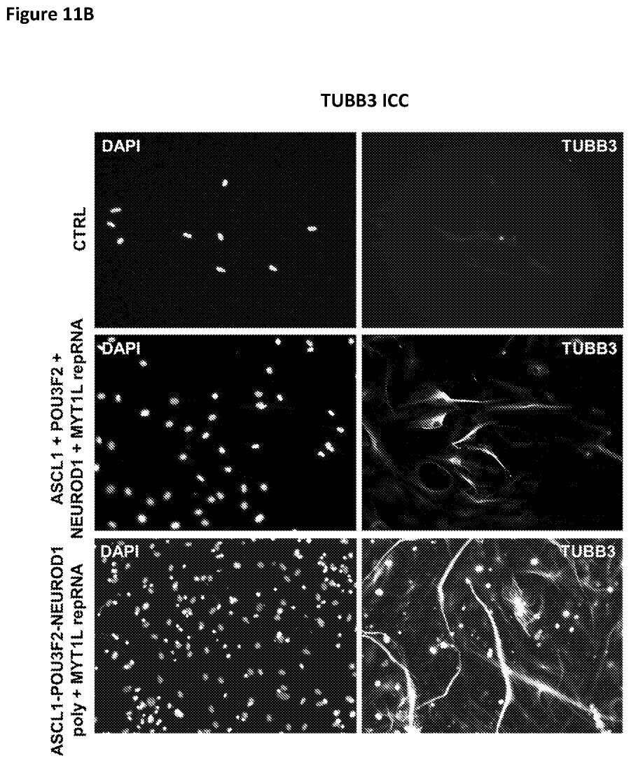

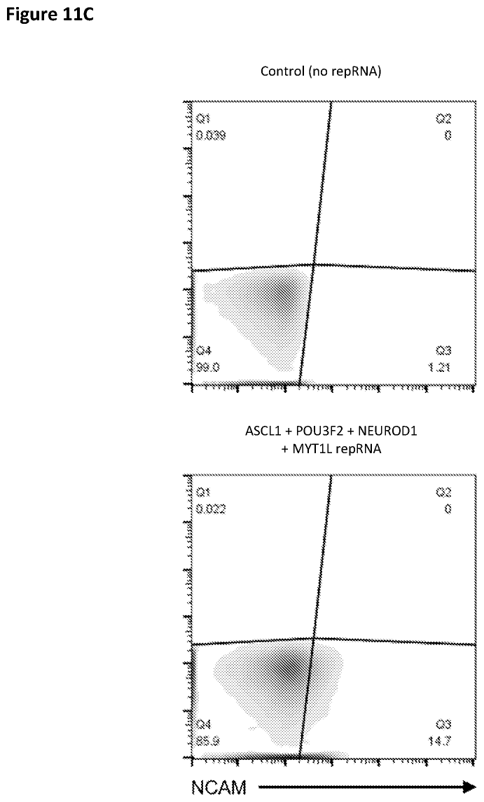

For dermal fibroblast to neuron conversion experiments, HDFs were co-transfected with ASCL1, POU3F2, NEUROD1 and MYT1L repRNA in equimolar amounts or with a polycistronic ASCL1-POU3F2-NEUROD1 repRNA with MYT1L repRNA, as previously described, and plated onto Geltrex (Thermo Fisher) coated culture plates. To obtain ASCL1, POU3F2, NEUROD1 and MYTIL repRNA, separate plasmid templates were constructed, with sequence encoding for either ASCL1, POU3F2, NEUROD1 or MYT1L inserted at the multiple cloning site of the self-replicating RNA vector. To obtain the ASCL1-POU3F2-NEUROD1 polycistronic repRNA, a plasmid template was constructed by Gibson assembly, inserting sequence encoding ASCL1-P2A-POU3F2-E2A-NEUROD1 into the multiple cloning site of the self-replicating RNA vector (see FIG. 11A). These plasmids were used as templates to generate ASCL1, POU3F2 (BRN2), NEUROD1, MYT1L and ASCL1-POU3F2-NEUROD1 polycistronic repRNA by in vitro transcription. Following co-transfection, cells were seeded in dermal fibroblast growth media which was replaced completely at 24 hour with neural induction media, consisting of 1:1 mix of DMEM:F12 and Neurobasal A supplemented with 2% B27 supplement, 0.5% N2 supplement, 500 μM valproic acid, 200 nM L-ascorbic acid, 10 μM Y-27632, +500 μM dBcAMP, 2 μM CHIR99021, 10 μM SB431542, 0.5 M LDN193189, 100 ng/ml noggin +10 μM ruxolitinib, with media changes every 2-3 days. Flow cytometry for NCAM1 expression and ICC analysis for TUBB3 expression were performed at day 14 post-transfection as previously described (FIG. 11).

Analysis of differentiated cultures showed ASCL1, POU3F2, NEUROD1 and MYT1L, when introduced as individual repRNA or partly encoded by a polycistronic repRNA, were capable of converting dermal fibroblasts to neurons. ICC analysis of TUBB3 expression detects neuronal cells with expected class III β-tubulin cellular distribution in cultures of HDF electroporated with either ASCL1, POU3F2, NEUROD1 and MYT1L individual repRNA or with a polycistronic ASCL1-POU3F2-NEUROD1 repRNA with MYT1L repRNA (FIG. 11B). Example flow cytometry to detect the neuronal cell surface marker NCAM, confirms neuronal marker expression in converted HDF cultures (FIG. 11C).

These data demonstrate that repRNA encoding specific transcription factors can convert dermal fibroblasts to neurons, and that at least part of the transcription factor combination can be successfully encoded in a polycistronic repRNA. This further illustrates the broad applicability of using repRNA for cell conversions across different starting source cell types.

### Example 8: Conversion of iPSC and Dermal Fibroblasts to Myocytes Using MYOD1-expressing Self-replicating RNA

Conversion of both iPSC and fibroblasts to myocytes can be achieved by expression of MyoD/MYOD1 as a single transcription factor and represents a further model system to validate the ability of repRNA to drive cell conversions (Abujarour et al., 2014 Stem Cells Translational Medicine, 3(2), 149-160; doi.org/10.5966/sctm.2013-0095; Choi et al., 1990 PNAS, 87(20), 7988-7992; doi.org/10.1073/pnas.87.20.7988).

To further test the ability of repRNA to drive cell conversions, a plasmid template was constructed, with sequence encoding MYOD1 inserted at the multiple cloning site of the self-replicating RNA vector. This plasmid was used as a template to generate MYOD1 repRNA by in vitro transcription. For iPSC to myocyte conversion experiments, iPSC were nucleofected with MYOD1 repRNA as previously described, and plated onto Geltrex-coated culture plates. Media was changed 24 hours post-nucleofection, to αMEM+5% Knockout Serum Replacement+1 mM sodium pyruvate+50 μM 2-mercaptoethanol (Thermo Fisher)+1 μM all-trans retinoic acid (Sigma) with media changes every 2-3 days. For fibroblast to myocyte conversion experiments, HDFs were transfected with MYOD1 repRNA as previously described, and plated onto Geltrex-coated culture plates. Following transfection, cells were seeded directly into DMEM+10% foetal calf serum+8 μg/mL insulin (Thermo Fisher)+0.1 μM LDN-193189 (Tocris)+1 μM RN-1 (Tocris) (Cacchiarelli et al., 2018 Cell Systems, 7(3), 258-268.e3. https://doi.org/10.1016/j.cels.2018.07.006)+10 μM ruxolitinib, with media changes every 2-3 days. At day 7-10 post-transfection, cells were analysed by ICC. Following PBS wash, fixation and blocking as previously described, cells were incubated for 3 hour at room temperature with purified anti myosin heavy chain (MyHC) antibody (1:100; R&D Systems) in PBS+4% normal goat serum, and then 1 hour at room temperature with Alexa Fluor® 647-conjugated secondary antibody (1:1000; Thermo Fisher) in PBS+4% normal goat serum. Following antibody steps, cells were stored in PBS+1% BSA at 4° C. before imaging on a Leica DMi1 Inverted Microscope.

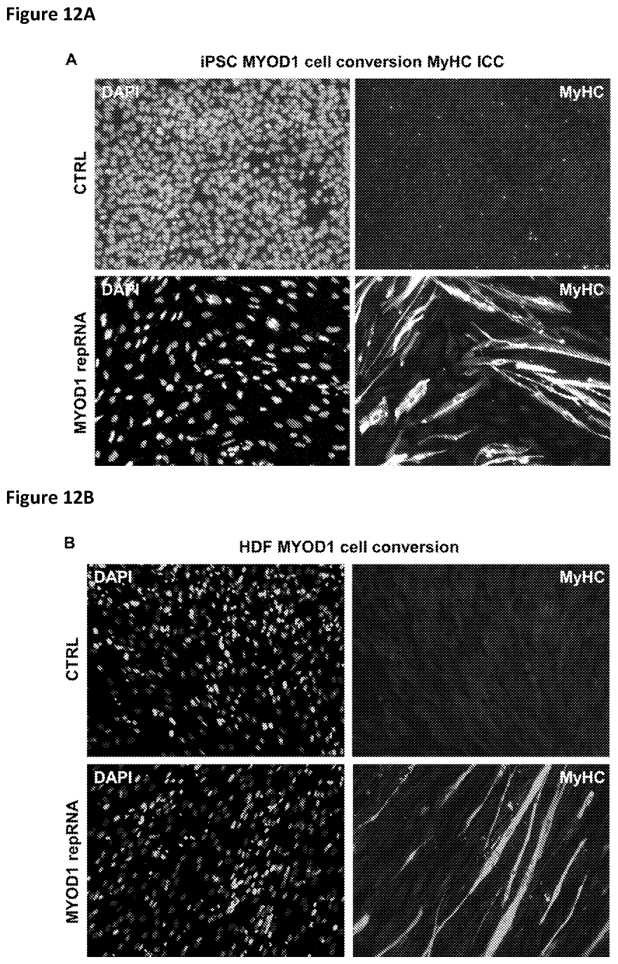

Analysis showed repRNA-encoded MYOD1 can convert both iPSC and HDF to myocytes at high efficiency. MyHC ICC indicated the presence of myocytes in cultures of MYOD1 repRNA transfected iPSC and HDF at day 7-10 post-transfection (FIG. 12A and FIG. 12B). This is a further example of the broad applicability of using repRNA for cell conversions across different source cell types and to obtain different differentiated target cell types.

Example 9: Conversion of Dermal Fibroblasts to Macrophage-like Cells Using SPI1 and CEBPA-expressing Self-replicating RNA

Conversion of fibroblasts to macrophage-like cells can be achieved by expression of the transcription factors SPI1 and CEPBA in dermal fibroblasts (Feng et al., 2008 PNAS, 105(16), 6057-6062; doi.org/10.1073/pnas.0711961105). To further exemplify the utility of self-replicating RNA for cell conversions, SPI1 and CEPBA expressing self-replicating RNA were tested for their ability to convert HDFs to macrophage-like cells.

For dermal fibroblast to macrophage-like cell conversion experiments, HDFs were co-transfected with SPI1 and CEBPA repRNA in equimolar amounts as previously described, and plated onto Geltrex

(Thermo Fisher) coated culture plates. To obtain SPI1 and CEBPA repRNA, separate plasmid templates were constructed, with sequence encoding for either SPI1 or CEPBA inserted at the multiple cloning site of the self-replicating RNA vector. These plasmids were used as templates to generate SPI1 and CEPBA repRNA by in vitro transcription. Following co-transfection, cells were seeded directly into DMEM +10% foetal calf serum +10 ng/ml M-CSF (Peprotech)+10 μM ruxolitinib, with media changes every 2-3 days. Flow cytometry and qPCR analysis was performed at day 10 post-transfection as previously described. For flow cytometry, expression of the leukocyte common antigen, CD45, was detected using a Brilliant Violet 650™ conjugated anti-human CD45 mouse monoclonal antibody (1:300; BioLegend), and expression of macrophage-1 antigen (Mac-1), CD11b, was detected using a PE conjugated anti-human CD11b mouse monoclonal antibody (1:100, BioLegend). qPCR was performed as previously described, using pre-designed TaqMan assays to CSF2RB, CSF1R and LYZ.

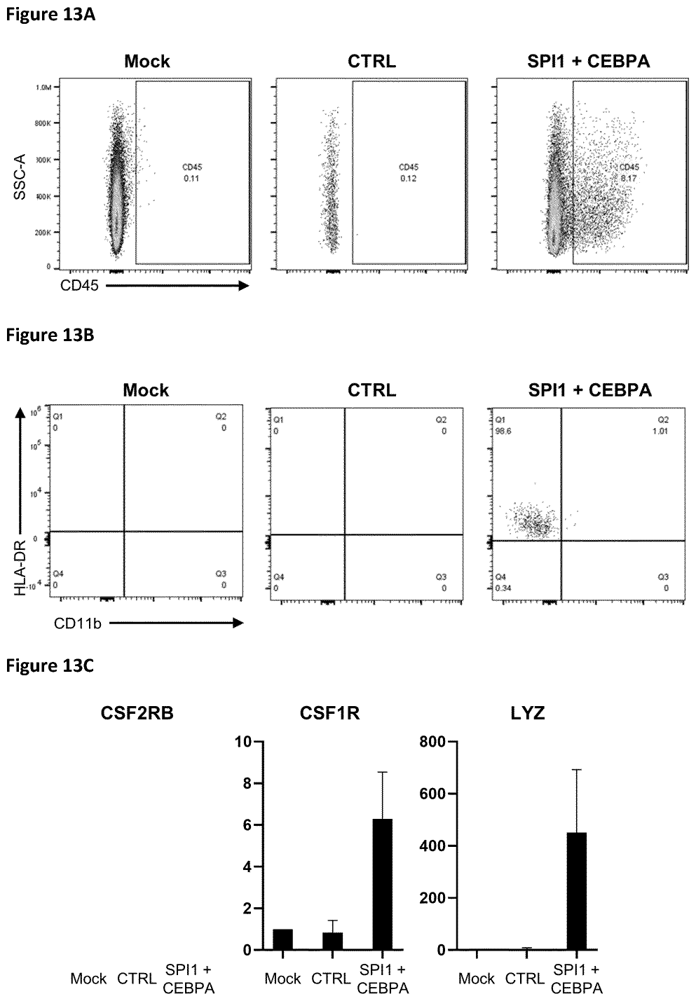

Analysis showed that co-transfection of HDFs with repRNA-encoded SPI1 and CEBPA can direct dermal fibroblasts to macrophage-like cells. Flow cytometry analysis showed significant expression of the leukocyte common antigen, CD45, in HDFs co-transfected with SPI1 and CEBPA repRNA compared to control mock and GFP repRNA transfected cultures that showed no CD45 expression (FIG. 13A). Within the CD45+ positive population in SPI1 and CEPBA repRNA co-transfected cells, the majority of cells express HLA-DR, a marker of antigen presenting cells, of which macrophages are one subtype. A proportion of cells also express macrophage specific CD11b (FIG. 13B). Further, assessment of gene expression by qPCR showed that SPI1 and CEBPA repRNA co-transfected HDFs express macrophage-associated genes, including CSF2RB, CSF1R and LYZ (FIG. 13C). This is a further example of the broad applicability of using repRNA for cell conversions to obtain different differentiated target cell types.

### Example 10: Conversion of Endothelial Cells to Hepatic Progenitor Cells Using HNF1A, HNF4A, ONECUT1 and FOXA3-expressing Self-replicating RNA

Conversion of endothelial cells to hepatic progenitor cells can be achieved by expression of the transcription factors HNF1A, HNF4A, ONECUT1 and FOXA3 in endothelial cells (Inada et al., 2020 Nature Communications, 11(1), 1-17; doi.org/10.1038/s41467-020-19041-z). To further exemplify the utility of self-replicating RNA for cell conversions, HNF1A, HNF4A, ONECUT1 and FOXA3 expressing self-replicating RNA were tested for their ability to convert human umbilical vein endothelial cells (HUVECs) to hepatic progenitor cells.

Human umbilical vein endothelial cells (HUVECs; ATCC) were routinely maintained in complete EGM2 endothelial growth media (Lonza) and passaged at a 1:3-1:5 ratio every 3-4 days. For nucleofection,

HUVECs were treated for 5 min with TrypLE to make a single cell suspension, collected and counted. Required numbers of cells were centrifuged for 4 min at 300 g and resuspended in complete P5 buffer (P5 Primary Cell Nucleofector Kit; Lonza). HUVECs were nucleofected using the Lonza 4D system with 5 μg total repRNA per 1×106 cells. Nucleofected cells were plated in complete EGM2 endothelial growth media.

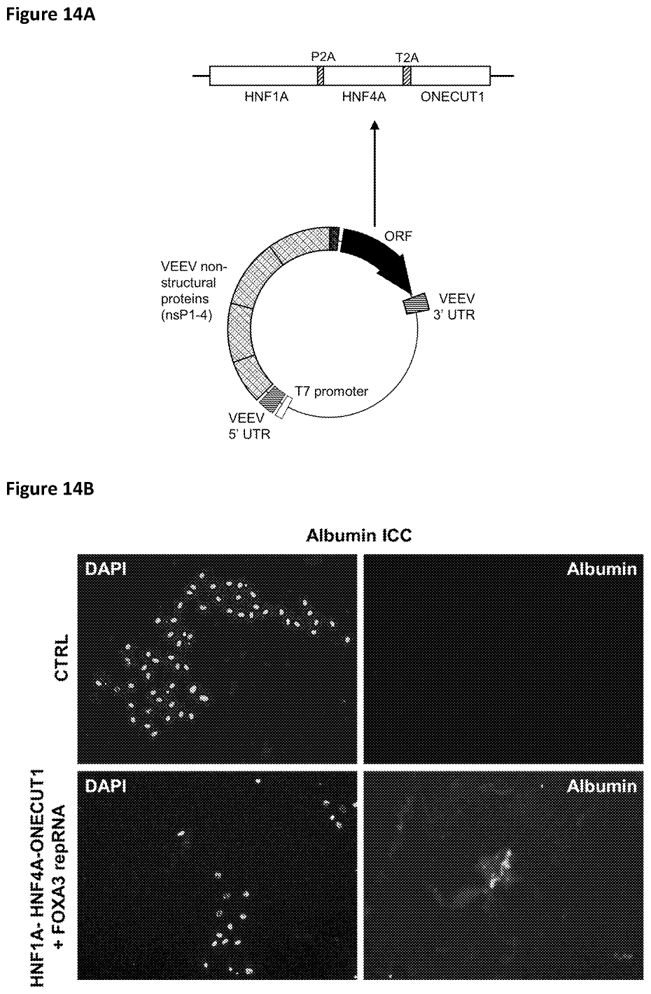

For endothelial cell to hepatic progenitor cell conversion experiments, HUVECs were co-transfected with a HNF1A-HNF4A-ONECUT1 polycistronic repRNA and a FOXA3 repRNA in equimolar amounts, and plated onto Geltrex-coated culture plates. To obtain FOXA3 repRNA, a plasmid template was constructed, with sequence encoding for FOXA3 inserted at the multiple cloning site of the self-replicating RNA vector. To obtain the HNF1A-HNF4A-ONECUT1 polycistronic repRNA, a plasmid template was constructed by Gibson assembly, inserting sequence encoding HNF1A-P2A-HNF4A-T2A-ONECUT1 into the multiple cloning site of the self-replicating RNA vector (see FIG. 14A). These plasmids were used as templates to generate FOXA3 and polycistronic HNF1A-P2A-HNF4A-T2A-ONECUT1 repRNA by in vitro transcription. Following co-transfection, cells were seeded in complete EGM2 endothelial growth media +200 ng/ml B18R, which was replaced completely at 72 hour with hepatocyte growth media consisting of hepatocyte culture medium (Lonza) supplemented with 100 nM dexamethasone, 10 mM nicotinamide, 1 μM A83-01, 2 μM SB431542, 5 μM Y-27632+200 ng/mL B18R, with media changes every 2-3 days. ICC was performed at day 14 post-transfection. Following PBS wash, fixation and blocking as previously described, cells were incubated overnight at room temperature with purified anti albumin antibody (1:500; Bethyl Laboratories) in PBS+4% normal donkey serum, and then 1 hour at room temperature with Alexa Fluor® 594-conjugated secondary antibody (1:1000; Thermo Fisher) in PBS +4% donkey goat serum. Following antibody steps, cells were stored in PBS+1% BSA at 4° C. before imaging on a Leica DMi1 Inverted Microscope (FIG. 14B).

Analysis showed repRNA-encoded FOXA3, HNF1A, HNF4A and ONECUT1 can convert HDF to hepatic progenitor cells. Albumin ICC indicated the presence of hepatocytes in cultures of repRNA transfected HDF at day 14 post-transfection (FIG. 14B). This is a further example of the broad applicability of using repRNA for cell conversions across different source cell types, validating the applicability of using repRNA to obtain progenitor cells.

### Example 11: Different Approaches to Improve Expression of Self-replicating RNA in Dermal Fibroblasts to Enhance Cell Conversion

As stated earlier, it has previously been demonstrated that vaccinia virus-derived B18R protein and the small molecule JAK1/2 inhibitor Ruxolitinib are able to improve the expression dynamics of self-replicating RNA (Blakney et al., 2021). There are a number of potential ways in which to inhibit the cellular antiviral response to self-replicating RNA.

Different approaches to inhibiting the cellular antiviral response and improving the expression of self-replicating RNA in dermal fibroblasts, were tested by assessing their ability to improve GFP expression from a GFP repRNA and enhance the MYOD1 repRNA-mediated conversion of HDFs to myocytes. These included further small molecule JAK pathway inhibitors (e.g. fedratinib, upadacitinib, filgotinib, baricitinib, deucravacitinib, ritlecitinib, decernotinib), RIG-I inhibitors (e.g. RIG012), TBK1 inhibitors (e.g. BX795, MRT67307), viral-derived interferon decoys (e.g. Y136R) and recombinant protein inhibitors genetically encoded within self-replicating RNA (e.g. genetically encoded B18R).

For these experiments, GFP-IRES-Puro^{R} and MYOD1 repRNA were transfected into HDFs as previously described, and for testing genetically encoded B18R, B18R repRNA was co-transfected at equimolar amounts with either GFP-IRES-Puro^{R} or MYOD1 repRNA. To obtain B18R repRNA, a plasmid template was constructed, with sequence encoding for the B18R gene inserted at the multiple cloning site of the self-replicating RNA vector. This plasmid was used as a template to generate B18R repRNA by in vitro transcription.

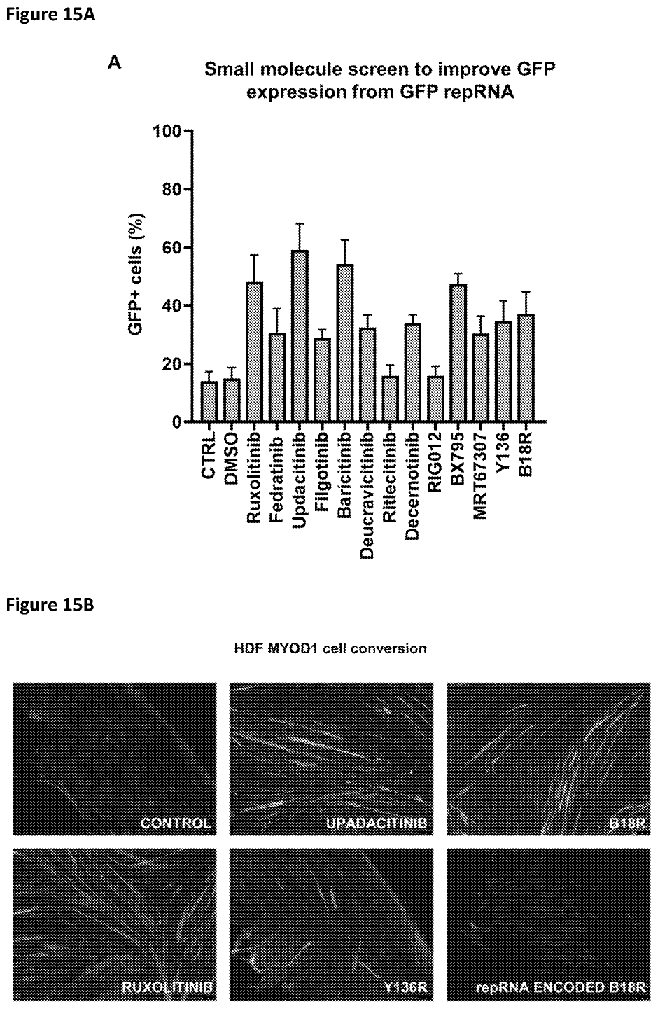

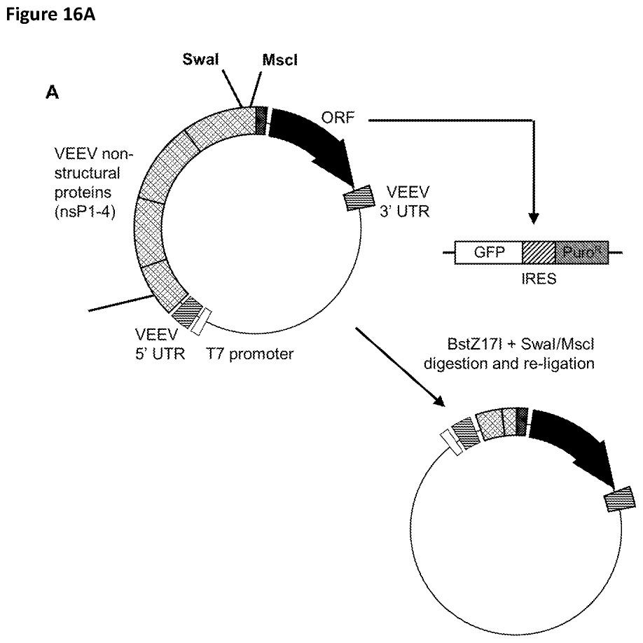

To assess the effect of inhibitors of cellular antiviral response on maintenance of GFP repRNA, HDFs were transfected a with GFP-IRES-Puro^{R} repRNA, and then seeded directly into growth media, with the addition of various small molecules and recombinant proteins. Cells were media changed every 2-3 days and assessed for GFP expression by flow cytometry at day 7 post-transfection. Media was supplemented with small molecules including 5 μM ruxolitinib, 2 μM fedratinib, 5 μM upadacitinib, 5 μM filgotinib, 5 μM baricitinib, 5 μM deucravacitinib, 5 μM ritlecitinib, 5 μM decernotinib, 5 μM BX795, 5 μM MRT67307 (all Selleckchem), 1 μM RIG012 (Axonmedchem), 200 ng/mL B18R or 200 ng/ml Y136R (BioTechne). A condition with DMSO supplemented media was included as a control. Flow cytometry analysis showed that a number of treatments were able to maintain an increased percentage of GFP positive cells over control treatments (no media addition or DMSO addition) and at a similar level to the routinely used inhibitors ruxolitinib and B18R recombinant protein (FIG. 15A).

To assess the effect of inhibitors of cellular antiviral response on maintenance of MYOD1 repRNA, fibroblast to myocyte conversion experiments were set up as previously described, and then seeded directly into growth media, with the addition of various small molecules and recombinant proteins. Small molecule and recombinant protein additions were maintained in all subsequent media changes, and at day 7-10 post-transfection, cell conversion was assessed by MyHC ICC as previously described. Analysis indicated that as well as ruxolitinib and B18R, further small molecule (e.g. upadacitinib) and recombinant protein (e.g. Y136R) inhibitors of the cellular antiviral response are capable of maintaining repRNA expression to a sufficient level to permit cell conversion (FIG. 15B). As observed previously, control cultures of fibroblasts transfected with MYOD1 repRNA and maintained in standard media not supplemented with an inhibitor of the cellular antiviral response failed to convert to skeletal myocytes (FIG. 15B—control).

Together these data demonstrate multiple different ways to modulate the host innate immune response to repRNA, to prolong repRNA expression to sufficient levels to drive cell conversion.

### Example 12: Trans-amplifying RNA

Self-replicating RNA can comprise two RNA molecules: a first molecule comprising sequences encoding non-structural proteins required for replication of the RNA, and a second molecule comprising a sequence encoding at least one transcription factor, to provide a trans-replicating RNA system.

The non-structural proteins of alphavirus-derived self-replicating RNAs assemble to generate a multi-enzyme replicase complex, which is capable of recognizing sequence elements within the self-replicating RNA replicon and replicating the entire RNA template. The replicase complex is also capable of replicating full-length and truncated RNAs in trans, and minimally requires a series of conserved sequence elements in these “trans-replicons” for their replication (Beissert et al., 2019)). The replicase activity can be supplied in a number of ways, including by delivery of a plasmid, an mRNA or a separate self-replicating RNA encoding the required non-structural proteins.

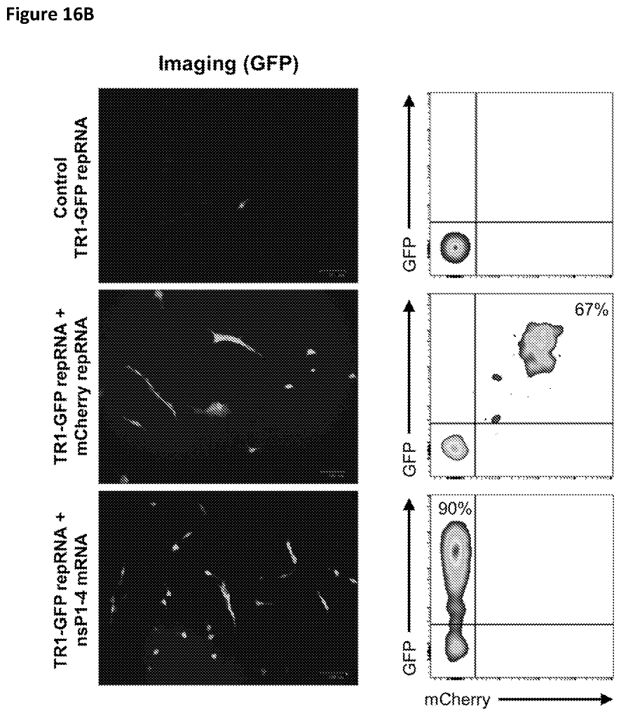

To assess the applicability of a trans-replicating RNA system, trans-replicon constructs were developed. Utilizing restriction enzyme sites within the non-structural proteins of the self-replicating RNA plasmid, two non-structural protein deletion constructs were generated. TR1 GFP plasmid template was generated by re-ligation of the previously described GFP-IRES-Puro^{R} self-replicating RNA vector after BstZ171 +Swal digestion, to leave a trans-replicon deleted between the 5′ 503 bp of nsP1 and the 3′ 603 bp of nsP4. TR2 GFP plasmid template was generated by re-ligation after BstZ171 +Mscl digestion, to leave a trans-replicon deleted between the 5′ 503 bp of nsP1 and the 3′ 116 bp of nsP4 (FIG. 16A). These plasmids were used as templates to generate TR1 GFP and TR2 GFP repRNA by in vitro transcription. To obtain mCherry repRNA, a plasmid template was constructed, with sequence encoding for mCherry inserted at the multiple cloning site of the self-replicating RNA vector. This plasmid was used as a template to generate mCherry repRNA by in vitro transcription. Non-structural protein 1-4 mRNA was produced by in vitro transcription with a PCR template as previously described, generated by using GFP-IRES-Puro^{R} self-replicating RNA plasmid DNA as template and the primers detailed (SEQUENCE ID NO: 8 and 9).

| | |
|---|---|
| nsP1-4 mRNA | GCTAATACGACTCACTATAAGGACTCACTATTTGTTTTCGCGCCCAGTTGCAAAAAGTGT |
| template Primer 1 | CGCCACCATGGAGAAAGTTCACGTTGACAT (Sequence ID NO: 8) |
| nsP1-4 mRNA | ttttttttttttttttttttttttttttttttttttttttttttttttttttttttttttttttttttttttt |
| template Primer 2 | tttttttttttttttttttttttttttttttttttttttttttttttTTAGCCGTAGAGAGTTATAGGGGC |
| | (Sequence ID NO: 9) |

iPSC were co-nucleofected with equimolar amounts of either TR1 GFP or TR2 GFP repRNA in combination with mCherry repRNA or nsP1-4 mRNA, and plated and maintained as previously described, with images taken at 48 hr and flow cytometry analysis performed at 96 hr post-nucleofection.

Analysis showed that transfection of iPSC with just GFP-expressing trans-replicons lacking a functional replicase, resulted in no GFP expression (FIG. 16B). Co-transfection of iPSC with the GFP-expressing trans-replicons, and a source of replicase, encoded either by an mCherry repRNA or nsP1-4 mRNA, resulted in functional GFP expression from the trans-replicon, demonstrating a functional trans-replicon system. Imaging at 48 hr and flow cytometry analysis at 96 hr showed no GFP expression in iPSC transfected with either TR1-GFP or TR2-GFP repRNA alone, but when TR1-GFP or TR2-GFP were co-transfected with either mCherry repRNA or nsP1-4 mRNA, high levels of GFP expression were observed.

Together these data show that, in principle, the replicative capacity of repRNA driven by an nsP1-4 encoded replicase, can be split between two molecules. This allows truncation of the repRNA to a short trans-replicon, that is capable of being replicated and its cargo expressed, upon simultaneous addition of the replicase activity from another repRNA or nsP1-4 encoding mRNA.

1. A self-replicating RNA composition comprising a self-replicating RNA comprising a sequence encoding at least one transcription factor that converts a pluripotent stem cell, a multipotent stem cell, or a differentiated source cell to a differentiated target cell exhibiting at least one phenotypic characteristic of the differentiated target cell.

2. A self-replicating RNA composition comprising two RNA molecules: a first molecule comprising sequences encoding non-structural proteins required for replication of the RNA, and a second molecule comprising a sequence encoding at least one transcription factor that converts a pluripotent stem cell, a multipotent stem cell, or a differentiated source cell to a differentiated target cell exhibiting at least one phenotypic characteristic of the differentiated target cell.

3. The self-replicating RNA composition as claimed in claim 1 or 2, wherein the at least one transcription factor does not include more than one transcription factor from the list of known pluripotency inducing transcription factors consisting of POU5F1; SOX2; KLF4; MYC, MYCL, LIN28, NANOG; and GLIS1.

4. The self-replicating RNA composition as claimed in any of claims 1 to 3, wherein the transcription factor is selected from the transcription factors listed in Table 1 or 2 or 3.

5. The self-replicating RNA composition as claimed in any of claims 1 to 4, wherein the self-replicating RNA encodes two or more transcription factors.

6. The self-replicating RNA composition as claimed in any of claims 1 to 5, wherein the self-replicating RNA comprises:
(a) sequences encoding non-structural proteins capable of directing replication of the self-replicating RNA when delivered into a cell;
(b) cis-active replication sequences at the 5′ and 3′ ends of the RNA; and
(c) a promoter that can direct expression of an exogenous sequence.

7. The self-replicating RNA composition as claimed in any of claims 1 to 6, wherein the RNA is derived from a single stranded RNA virus.

8. The self-replicating RNA composition as claimed in claim 7, wherein the self-replicating RNA is derived from a single stranded RNA virus selected from the group consisting of alphavirus; picornavirus; flavivirus; rubivirus; pestivirus; hepacivirus; filovirus; calicivirus; and coronavirus.

9. The self-replicating RNA composition as claimed in claim 7 or 8, wherein the self-replicating RNA is derived from an alphavirus selected from the group consisting of: Semliki Forest Virus, Sindbis Virus, and Venezuelan Equine Encephalitis Virus.

10. The self-replicating RNA composition as claimed in any of claims 1 to 9, wherein the self-replicating RNA additionally comprises the elements selected from the list comprising: reporter genes, immune regulation genes, micro RNAs, long noncoding RNAs, antibiotic resistance genes, sequences encoding an inhibitor of type I and/or type Ill interferon responses or combination thereof.

11. The self-replicating RNA composition as claimed in claim 10, wherein the inhibitor of type I and/or type III interferon response is selected from the list comprising: the vaccinia virus proteins E3, K3, and B18R, the influenza virus protein NS1, and the small molecule inhibitors Ruxolitinib and Upadacitinib.

12. A self-replicating RNA as set forth in the RNA compositions of claims 1-11.

13. A DNA sequence encoding the self-replicating RNA of claim 12.

14. An expression cassette comprising a nucleic acid of claim 13.

15. A method of producing the self-replicating RNA of claim 12, the method comprising transcribing the DNA sequence of claim 13 in vitro in the presence of an RNA polymerase.

16. A self-replicating RNA formulation comprising the self-replicating RNA composition as claimed in any of claims 1 to 11, the self-replicating RNA as claimed in claim 12 or the DNA sequence as claimed in claim 13, and a lipid or a lipid nanoparticle.

17. A method for converting a source cell to a cell exhibiting at least one phenotypic characteristic of a differentiated target cell, wherein the source cell does not exhibit the phenotypic characteristic of the differentiated target cell, the method comprising introducing the self-replicating RNA composition as claimed in any of claims 1 to 11, the self-replicating RNA as claimed in claim 12, the DNA sequence as claimed in claim 13 or the formulation as claimed in claim 16, into a source cell.

18. A method of generating a population of differentiated target cells from a population of source cells, the method comprising introducing a self-replicating RNA encoding at least one transcription factor into said source cells, thereby increasing the amount of the at least one transcription factor in the source population and maintaining the source cells under conditions to allow at least 0.1% of the source cells to be converted into cells exhibiting at least one phenotypic characteristic of the differentiated target cell.

19. The method as claimed in claim 17 or 18, wherein at least one phenotypic characteristic of the differentiated target cell is the upregulation of any one or more differentiated target cell markers and/or a change in cell morphology to more closely resemble the differentiated target cell and/or exhibiting the individual cell function of the differentiated target cell.

20. The method as claimed in any of claims 17 to 19, wherein the source cell is in a living animal, and the method comprises administering the self-replicating RNA composition as claimed in any of claims 1 to 11, the self-replicating RNA as claimed in claim 12, the DNA sequence as claimed in claim 13 or the formulation as claimed in claim 16 to the animal, preferably by local administration to the cell in the animal.

21. The method of claim 20, wherein the animal is a mammal, preferably a human.

22. The method of any of claims 17 to 21, wherein the source cell is in vitro.

23. A method of treating a patient in need thereof comprising administering the self-replicating RNA composition as claimed in any of claims 1 to 11, the self-replicating RNA as claimed in claim 12, the DNA sequence as claimed in claim 13 or the formulation as claimed in claim 16, to said patient.

24. Use of the self-replicating RNA composition as claimed in any of claims 1 to 11, the self-replicating RNA as claimed in claim 12, the DNA sequence as claimed in claim 13 or the formulation as claimed in claim 16, for the treatment of disease.

25. The self-replicating RNA composition as claimed in any of claims 1 to 11, the self-replicating RNA as claimed in claim 12, the DNA sequence as claimed in claim 13 or the formulation as claimed in claim 16, for use in medicine.


SEQUENCE LISTING (9 sequences)

SEQ ID NO: 1 - DNA, 45 bp, Artificial Sequence
  26S subgenomic promoter
gggcccctataactctctacggctaacctgaatggactacgacat

SEQ ID NO: 2 - protein, 19 aa, Artificial Sequence
  Porcine teschovirus-I 2A (P2A)
ATNFSLLKQAGDVEENPGP

SEQ ID NO: 3 - protein, 20 aa, Artificial Sequence
  Thosea asigna virus 2A (T2A)
QCTNYALLKLAGDVESNPGP

SEQ ID NO: 4 - protein, 22 aa, Artificial Sequence
  Foot and mouth disease virus (F2A)
VKQTLNFDLLKLAGDVESNPGP

SEQ ID NO: 5 - DNA, 531 bp, Artificial Sequence
  IRES encoding sequence
gctagcaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataat

SEQ ID NO: 6 - DNA, 88 bp, Artificial Sequence
  Primer 1
gctaatacgactcactataaggactcactatttgttttcgcgcccagttgcaaaaagtgtcgccaccatggtgagcaagggcgaggag

SEQ ID NO: 7 - DNA, 144 bp, Artificial Sequence
  Primer 2
ttttttttttttttttttttttttttttttttttttttttttttttttttttttttttttttttttttttttttttttttttttttttttttttttttttttttttttttttttttttttttacttgtacagctcgtccatgcc

SEQ ID NO: 8 - DNA, 90 bp, Artificial Sequence
  nsP1-4 mRNA template Primer 1
gctaatacgactcactataaggactcactatttgttttcgcgcccagttgcaaaaagtgtcgccaccatggagaaagttcacgttgacat

SEQ ID NO: 9 - DNA, 144 bp, Artificial Sequence
  nsP1-4 mRNA template Primer 2
ttttttttttttttttttttttttttttttttttttttttttttttttttttttttttttttttttttttttttttttttttttttttttttttttttttttttttttttttttttttttttagccgtagagagttataggggc